(19) United States
(12) Patent Application Publication
(10) Pub. No.: US 2021/0324418 A1
(43) Pub. Date: Oct. 21, 2021

(54) RECOMBINANT VIRAL VECTORS AND NUCLEIC ACIDS FOR PRODUCING THE SAME

(71) Applicant: StrideBio, Inc., Durham, NC (US)

(72) Inventors: Darby Thomas, Raleigh, NC (US); David Dismuke, Cary, NC (US)

(21) Appl. No.: 17/326,884

(22) Filed: May 21, 2021

Related U.S. Application Data
(63) Continuation of application No. PCT/US2019/062531, filed on Nov. 21, 2019.
(60) Provisional application No. 62/770,202, filed on Nov. 21, 2018.

(51) Int. Cl. C12N 15/86; C07K 14/47; A61K 9/00

(52) U.S. Cl. CPC C12N 15/86; C12N 2750/14143; A61K 9/0085; C07K 14/47

(57) ABSTRACT

Described herein are nucleic acids, AAV transfer cassettes and plasmids used in the production of recombinant adeno-associated viral (rAAV) vectors. The disclosed nucleic acids, cassettes and plasmids comprise sequences that express one or more transgenes having therapeutic efficacy in the amelioration, treatment and/or prevention of one or more diseases or disorders.

### CROSS REFERENCE TO RELATED APPLICATION

This application is a continuation of International Application No. PCT/US2019/062531, filed on Nov. 21, 2019, which claims priority to U.S. Provisional Application No. 62/770,202, filed on Nov. 21, 2018. The contents of these applications are incorporated by reference herein in their entirety for all purposes.

### FIELD

The instant disclosure relates to the fields of molecular biology and gene therapy. More specifically, disclosure relates to compositions and methods for producing recombinant viral vectors.

### DESCRIPTION OF THE TEXT FILE SUBMITTED ELECTRONICALLY

The contents of the text file submitted electronically herewith are incorporated by reference in their entirety: a computer readable format copy of the Sequence Listing (filename: STRD-011-01US_Sequence_Listing.txt, date recorded May 21, 2021, file size ˜145 kilobytes).

### BACKGROUND

Recombinant viral vectors, including adeno-associated virus vectors (AAVs), are useful as gene delivery agents, and are powerful tools for human gene therapy. Using AAVs, high-frequency stable DNA integration and expression may be achieved in a variety of cells, in vivo and in vitro. Unlike some other viral vector systems, AAV does not require active cell division for stable integration in target cells.

Recombinant AAV vectors can be produced in culture using viral production cell lines. Production of recombinant AAVs typically requires the presence of three elements in the cells: 1) a nucleic acid comprising a transgene flanked by AAV inverted terminal repeat (ITR) sequences, 2) AAV rep and cap genes, and 3) helper virus protein sequences. These three elements may be provided on one or more plasmids, and transfected or transduced into the cells.

The production and use of recombinant AAV vectors has been limited by the inability to efficiently package transgene DNA into viral capsids and to effectively express the transgene in target cells. Accordingly, there exists a need in the art for improved compositions and methods for producing recombinant AAV vectors.

### SUMMARY

Described herein are nucleic acids comprising AAV transfer cassettes. The disclosed nucleic acids can be used in the production of recombinant adeno-associated viral (AAV) vectors. The disclosed nucleic acids and transfer cassettes comprise the sequences of one or more transgenes having therapeutic efficacy in the amelioration, treatment and/or prevention of one or more diseases or disorders.

In some embodiments, the disclosure provides a nucleic acid comprising, from 5′ to 3′, a 5′ inverted terminal repeat (ITR), a promoter, a transgene sequence, a polyadenylation signal, and a 3′ ITR. In some embodiments, the transgene sequence encodes the frataxin (FXN) protein. The FXN protein may be, for example, the human FXN protein. In some embodiments, the FXN protein has the sequence of SEQ ID NO: 65, or a sequence that is at least 95% identical thereto. In some embodiments, the nucleic acid comprises the sequence of any one of SEQ ID NO: 28-64, or a sequence at least 95% identical thereto.

In some embodiments, the 5′ ITR is the same length as the 3′ ITR. In some embodiments, the 5′ ITR and the 3′ ITR have different lengths. In some embodiments, at least one of the 5′ ITR and the 3′ ITR is about 110 to about 160 nucleotides in length. At least one of the 5′ ITR and the 3′ ITR may be isolated or derived from, for example, the genome of AAV1, AAV2, AAV3, AAV4, AAV5, AAV6, AAV7, AAV8, AAV9, AAV10, AAV11, AAV12, AAVrh8, AAVrh10, AAVrh32.33, AAVrh74, Avian AAV or Bovine AAV. In some embodiments, the 5′ ITR comprises the sequence of SEQ ID NO: 1, or a sequence at least 95% identical thereto. In some embodiments, the 3′ ITR comprises the sequence of SEQ ID NO: 2, or a sequence at least 95% identical thereto. In some embodiments, the 3′ ITR comprises the sequence of SEQ ID NO: 3, or a sequence at least 95% identical thereto.

The promoter may drive expression of the transgene. In some embodiments, the promoter is a constitutive promoter. In some embodiments, the promoter is an inducible promoter. In some embodiments, the promoter is a tissue-specific promoter. In some embodiments, the promoter is a modified form of a wildtype promoter. For example, because of the packaging restrictions for an AAV, the length of a promoter may be reduced. In some embodiments, the promoter is a truncated form of a wildtype promoter.

The promoter may, for example, the CMV promoter, the SV40 early promoter, the SV40 late promoter, the metallothionein promoter, the murine mammary tumor virus (MMTV) promoter, the Rous sarcoma virus (RSV) promoter, the polyhedrin promoter, the chicken β-actin (CBA) promoter, the EF-1 alpha promoter, the EF-1 alpha short promoter, the EF-1 alpha core promoter, the dihydrofolate reductase (DHFR) promoter, the GUSB240 promoter, the GUSB379 promoter, or the phosphoglycerol kinase (PGK) promoter. In some embodiments, the promoter comprises a sequence selected from any one of SEQ ID NO: 6-12, or a sequence at least 95% identical thereto.

In some embodiments, the transgene sequence is CpG optimized. In some embodiments, the transgene sequence comprises SEQ ID NO: 19 or 20, or a sequence that is at least 95% identical thereto.

In some embodiments, the nucleic acid comprises a Kozak sequence immediately 5′ to the transgene sequence. The Kozak sequence may comprise, for example, the sequence of SEQ ID NO: 17 or 18, or a sequence at least 95% identical thereto.

In some embodiments, the polyadenylation signal is selected from the polyadenylation signal of simian virus 40 (SV40), human α-globin, rabbit α-globin, human β-globin, rabbit β-globin, human collagen, polyoma virus, human growth hormone (hGH) and bovine growth hormone (bGH). In some embodiments, the polyadenylation signal comprises the sequence of any one of SEQ ID NO: 21-24, or a sequence at least 95% identical thereto.

In some embodiments, the nucleic acid further comprises an enhancer. The enhancer may be, for example, a CMV enhancer. In some embodiments, the enhancer comprises the sequence of SEQ ID NO: 4 or 5, or a sequence at least 95% identical thereto.

In some embodiments, the nucleic acid further comprises an intronic sequence. The intronic sequence may be, for example, a chimeric sequence or a hybrid sequence. In some embodiments, the intronic sequence comprises a sequence isolated or derived from one or more of the following genes: β-globin, chicken beta-actin, minute virus of mice, and human IgG. In some embodiments, the intronic sequence comprises the sequence of any one of SEQ ID NO: 13-16, or a sequence at least 95% identical thereto.

In some embodiments, the nucleic acid further comprises at least one stuffer sequence (e.g., 1, 2, 3, 4, or 5 stuffer sequences). In some embodiments, the at least one stuffer sequence comprises the sequence of any one of SEQ ID NO: 25-27, or a sequence at least 95% identical thereto.

Also provided herein is a vector (e.g., an AAV vector or plasmid) comprising a nucleic acid of the disclosure.

Also provided is a cell comprising a nucleic acid of the disclosure.

Also provided is a method of producing a recombinant AAV vector, the method comprising contacting an AAV producer cell with a nucleic acid or plasmid/bacmid of the disclosure. Also provided is a recombinant AAV vector produced by this method. The recombinant AAV vector may comprise a capsid protein from AAV1, AAV2, AAV3, AAV4, AAV5, AAV6, AAV7, AAV8, AAV9, AAV10, AAV11, AAV12, AAVrh8, AAVrh10, AAVrh32.33, AAVrh74, Avian AAV and Bovine AAV. In some embodiments, the AAV vector may comprise a capsid protein with one or more substitutions or mutations compared to a wildtype AAV capsid protein. In some embodiments, the recombinant AAV vector is single stranded (ssAAV). In some embodiments, the recombinant AAV vector is self-complementary (scAAV).

Also provided are compositions comprising a nucleic acid, a plasmid, a bacmid, a cell, or a recombinant AAV vector of the disclosure.

Also provided is a method for treating a subject in need thereof comprising administering to the subject a therapeutically effective amount of a nucleic acid, a plasmid, a cell, or a recombinant AAV vector of the disclosure. In some embodiments, the subject is a human subject. In some embodiments, the subject has Friedreich's Ataxia (FRDA).

These and other embodiments are addressed in more detail in the detailed description set forth below.

### BRIEF DESCRIPTION OF THE DRAWINGS

FIG. 1 shows AAV production yield (vector genomes) using a triple plasmid transfection method. AAV vectors were quantified using a droplet digital PCR (ddPCR®) assay.

FIG. 2 shows percent survival of FXN-deficient (FXN^{flox/flox}MCKCre⁺) mice treated with an AAV vector packaging a human FXN transgene at a dose of 5×10¹³ vg/kg (Group 2) compared to saline-injected mice (Group 1).

FIG. 3A-3C show the result of an experiment wherein 3 week old FXN-deficient (FXN^{flox/flox}MCKCre⁺) mice were treated with either saline or an AAV vector packaging a human FXN transgene (low dose=1×10¹³ vg/kg, high dose=5×10¹³ vg/kg). Mice were sacrificed 3 weeks after treatment. FIG. 3A shows the number of copies of human FXN vector DNA per microgram of host DNA in heart tissue. FIG. 3B shows the number of copies of FXN mRNA, normalized to HPRT (Hypoxanthine-guanine phospho-ribosyltransferase) mRNA. ND=not detected. FIG. 3C shows FXN protein levels.

FIG. 4 shows expression of human FXN (ng/mg) in cultured Lec2 cells transduced with various doses of AAV9-FXN. Human FXN levels were measured using a standard ELISA.

FIG. 5 shows a schematic of an exemplary scheme for producing AAV using an AAV transfer cassette of the disclosure. An AAV transfer cassette comprising a 5′ITR, a promoter, a transgene, and a 3′ITR is packaged into a plasmid using standard cloning techniques. A second plasmid comprising AAV rep and cap sequences, and third plasmid comprising Adenovirus helper genes is prepared. The three plasmids are transfected into an AAV producer cell line (e.g., HEK293). The cells then produce AAVs, which can be purified and frozen for later use.

### DETAILED DESCRIPTION

Gene therapy holds great promise for the treatment and prevention of genetic diseases and disorders including, for example, Friedreich's Ataxia (FRDA). FRDA is an autosomal recessive disorder typically caused by mutations in the frataxin (FXN) gene. About 1 in 50,000 people in the United States have FRDA. The typical age of onset is between about 5 and about 18 years. Symptoms vary among subjects, but may include (i) loss of coordination (ataxia) in the arms and legs, (ii) fatigue/energy deprivation and muscle loss, (iii) vision impairment, hearing loss, and slurred speech, (iv) aggressive scoliosis (curvature of the spine), (v) diabetes mellitus (typically insulin-dependent), and (vi) serious heart conditions (e.g., hypertrophic cardiomyopathy and arrhythmias). The mental capabilities of individuals with FRDA remain intact. There are currently no treatments for FRDA; subjects are monitored for symptom management. Accordingly, there is a need in the art for compositions and methods to treat and/or prevent FRDA.

Provided herein are nucleic acids comprising AAV transfer cassettes for producing AAV vectors. The AAV vectors can be used for gene therapy applications, for example to deliver a therapeutic transgene to a cell or to a subject in need thereof. The AAV transfer cassettes and vectors of the instant disclosure may be used to treat or prevent various genetic diseases and disorders, such as FRDA.

All papers, publications and patents cited in this specification are herein incorporated by reference as if each individual paper, publication or patent were specifically and individually indicated to be incorporated by reference and are incorporated herein by reference to disclose and describe the methods and/or materials in connection with which the publications are cited.

Unless the context indicates otherwise, it is specifically intended that the various features described herein can be used in any combination. Section headers are used herein for purposes of organization, and are not intended to be limiting.

Unless otherwise defined, all technical and scientific terms used herein have the same meaning as commonly understood by one of ordinary skill in the art to which this disclosure belongs. The terminology used in the detailed description herein is for the purpose of describing particular embodiments only and is not intended to be limiting.

### Definitions

The following terms are used in the description herein and the appended claims:

The singular forms “a,” “an” and “the” are intended to include the plural forms as well, unless the context clearly indicates otherwise.

Furthermore, the term “about” as used herein when referring to a measurable value such as an amount or the length of a polynucleotide or polypeptide sequence, dose, time, temperature, and the like, is meant to encompass variations of ±20%, ±10%, ±5%, ±1%, ±0.5%, or even ±0.1% of the specified amount.

Also as used herein, “and/or” refers to and encompasses any and all possible combinations of one or more of the associated listed items, as well as the lack of combinations when interpreted in the alternative (“or”).

A “nucleic acid” or “polynucleotide” is a sequence of nucleotide bases, for example RNA, DNA or DNA-RNA hybrid sequences (including both naturally occurring and non-naturally occurring nucleotides). In some embodiments, the nucleic acids of the disclosure are either single or double stranded DNA sequences. A nucleic acid may be 1-1,000, 1,000-10,000, 10,000-100,000, 100,000-1 million or greater than 1 million nucleotides in length. A nucleic acid will generally contain phosphodiester bonds, although in some cases nucleic acid analogs are included that may have alternate backbones, comprising, for example, phosphoramide, phosphorothioate, phosphorodithioate, O-methylphophoroamidite, or P-ethoxy linkages, or peptide nucleic acid backbones and linkages. Other analog nucleic acids include those with positive backbones, non-ionic backbones, and non-ribose backbones. Nucleic acids containing one or more carbocyclic sugars are also included within the definition of nucleic acids. These modifications of the ribose-phosphate backbone may facilitate the addition of labels, or increase the stability and half-life of such molecules in physiological environments. Nucleic acids of the disclosure may be linear, or may be circular (e.g., a plasmid).

The terms “protein,” “peptide,” and “polypeptide” are used interchangeably herein, and refer to a compound comprised of amino acid residues covalently linked by peptide bonds. A protein or peptide must contain at least two amino acids, but no limitation is placed on the maximum number of amino acids that can comprise a protein's or peptide's sequence.

As used herein, the terms “virus vector,” “viral vector,” or “gene delivery vector” refer to a virus particle that functions as a nucleic acid delivery vehicle, and which comprises the vector genome packaged within a virion. Exemplary virus vectors of the disclosure include adenovirus vectors, adeno-associated virus vectors (AAVs), lentivirus vectors, and retrovirus vectors.

Adeno-associated virus or AAV belongs to the Dependovirus genus of the Parvoviridae family. The 4.7 kb wildtype AAV genome encodes two major open reading frames. The rep gene expresses viral replication proteins and the cap gene expresses viral capsid proteins. At the ends of the AAV genome are inverted terminal repeats (ITRs) that form a T-shaped hairpin structure. Although the mature AAV virion is infectious in mammalian cells, the replicative AAV life cycle requires helper function from, for example, adenovirus or herpes virus. Recombinant AAV vectors can be generated by replacing the wildtype AAV open reading frames with a transgene expression cassette.

As described herein, an AAV may be AAV type 1, AAV type 2, AAV type 3 (including types 3A and 3B), AAV type 4, AAV type 5, AAV type 6, AAV type 7, AAV type 8, AAV type 9, AAV type 10, AAV type 11, AAV type 12, AAV type 13, AAV type rh32.33, AAV type rh8, AAV type rh10, AAV type rh74, AAV type hu.68, avian AAV, bovine AAV, canine AAV, equine AAV, ovine AAV, snake AAV, bearded dragon AAV, AAV2i8, AAV2g9, AAV-LK03, AAV7m8, AAV Anc80, AAV PHP.B, and any other AAV now known or later discovered. See, e.g., BERNARD N. FIELDS et al., VIROLOGY, volume 2, chapter 69 (4th ed., Lippincott-Raven Publishers). A number of AAV serotypes and clades have been identified (see, e.g., Gao et al, (2004) J. Virology 78:6381-6388; Moris et al, (2004) Virology 33-:375-383; and Table 1).

TABLE 1
AAV Serotypes and Clades

| | GenBank |
|---|---|
| Complete | Accession |
| Genomes | Number |
| Adeno- | NC_002077, |
| associated | AF063497 |
| virus 1 | |
| Adeno- | NC_001401 |
| associated | |
| virus 2 | |
| Adeno- | NC_001729 |
| associated | |
| virus 3 | |
| Adeno- | NC_001863 |
| associated | |
| virus 3B | |
| Adeno- | NC_001829 |
| associated | |
| virus 4 | |
| Adeno- | Y18065, |
| associated | AF085716 |
| virus 5 | |
| Adeno- | NC_001862, |
| associated | AAB95450.1 |
| virus 6 | |
| Avian AAV | AY186198, |
| ATCC | AY629583, |
| VR-865 | NC_004828 |
| Avian AAV | NC_006263, |
| strain DA-1 | AY629583 |
| Bovine AAV | NC_005889, |
| | AY388617, |
| | AAR26465 |
| AAV11 | AAT46339, |
| | AY631966 |
| AAV12 | ABI16639, |
| | DQ813647 |
| Clade A | |
| AAV1 | NC_002077, |
| | AF063497 |
| AAV6 | NC_001862 |
| Hu.48 | AY530611 |
| Hu 43 | AY530606 |
| Hu 44 | AY530607 |
| Hu 46 | AY530609 |
| Clade B | |
| Hu. 19 | AY530584 |
| Hu. 20 | AY530586 |
| Hu 23 | AY530589 |
| Hu22 | AY530588 |
| Hu24 | AY530590 |
| Hu21 | AY530587 |
| Hu27 | AY530592 |
| Hu28 | AY530593 |
| Hu 29 | AY530594 |
| Hu63 | AY530624 |
| Hu64 | AY530625 |
| Hu13 | AY530578 |
| Hu56 | AY530618 |
| Hu57 | AY530619 |
| Hu49 | AY530612 |
| Hu58 | AY530620 |
| Hu34 | AY530598 |
| Hu35 | AY530599 |
| AAV2 | NC_001401 |
| Hu45 | AY530608 |
| Hu47 | AY530610 |
| Hu51 | AY530613 |
| Hu52 | AY530614 |
| Hu T41 | AY695378 |
| Hu S17 | AY695376 |
| Hu T88 | AY695375 |
| Hu T71 | AY695374 |
| Hu T70 | AY695373 |
| Hu T40 | AY695372 |
| Hu T32 | AY695371 |
| Hu T17 | AY695370 |
| Hu LG15 | AY695377 |
| Clade C | |
| Hu9 | AY530629 |
| Hu10 | AY530576 |
| Hu11 | AY530577 |
| Hu53 | AY530615 |
| Hu55 | AY530617 |
| Hu54 | AY530616 |
| Hu7 | AY530628 |
| Hu18 | AY530583 |
| Hu15 | AY530580 |
| Hu16 | AY530581 |
| Hu25 | AY530591 |
| Hu60 | AY530622 |
| Ch5 | AY243021 |
| Hu3 | AY530595 |
| Hu1 | AY530575 |
| Hu4 | AY530602 |
| Hu2 | AY530585 |
| Hu61 | AY530623 |
| Clade D | |
| Rh62 | AY530573 |
| Rh48 | AY530561 |
| Rh54 | AY530567 |
| Rh55 | AY530568 |
| Cy2 | AY243020 |
| AAV7 | AY513851 |
| Rh35 | AY243000 |
| Rh37 | AY242998 |
| Rh36 | AY242999 |
| Cy6 | AY243016 |
| Cy4 | AY243018 |
| Cy3 | AY243019 |
| Cy5 | AY243017 |
| Rh13 | AY243013 |
| Clade E | |
| Rh38 | AY530558 |
| Hu66 | AY530626 |
| Hu42 | AY530605 |
| Hu67 | AY530627 |
| Hu40 | AY530603 |
| Hu41 | AY530604 |
| Hu37 | AY530600 |
| Rh40 | AY530559 |
| Rh2 | AY243007 |
| Bb1 | AY243023 |
| Bb2 | AY243022 |
| Rh10 | AY243015 |
| Hu17 | AY530582 |
| Hu6 | AY530621 |
| Rh25 | AY530557 |
| Pi2 | AY530554 |
| Pi1 | AY530553 |
| Pi3 | AY530555 |
| Rh57 | AY530569 |
| Rh50 | AY530563 |
| Rh49 | AY530562 |
| Hu39 | AY530601 |
| Rh58 | AY530570 |
| Rh61 | AY530572 |
| Rh52 | AY530565 |
| Rh53 | AY530566 |
| Rh51 | AY530564 |
| Rh64 | AY530574 |
| Rh43 | AY530560 |
| AAV8 | AF513852 |
| Rh8 | AY242997 |
| Rh1 | AY530556 |
| Clade F | |
| Hu14 | AY530579 |
| (AAV9) | |
| Hu31 | AY530596 |
| Hu32 | AY530597 |
| HSC1 | MI332400.1 |
| HSC2 | MI332401.1 |
| HSC3 | MI332402.1 |
| HSC4 | MI332403.1 |
| HSC5 | MI332405.1 |
| HSC6 | MI332404.1 |
| HSC7 | MI332407.1 |
| HSC8 | MI332408.1 |
| HSC9 | MI332409.1 |
| HSC11 | MI332406.1 |
| HSC12 | MI332410.1 |
| HSC13 | MI332411.1 |
| HSC14 | MI332412.1 |
| HSC15 | MI332413.1 |
| HSC16 | MI332414.1 |
| HSC17 | MI332415.1 |
| Hu68 | |
| Clonal | |
| Isolate | |
| AAV5 | Y18065, |
| | AF085716 |
| AAV 3 | NC_001729 |
| AAV 3B | NC_001863 |
| AAV4 | NC_001829 |
| Rh34 | AY243001 |
| Rh33 | AY243002 |
| Rh32 | AY243003 |
| Others | |
| Rh74 | |
| Bearded | |
| Dragon AAV | |
| Snake AAV | NC_006148.1 |

The term “self-complimentary AAV” or “scAAV” refers to a recombinant AAV vector which forms a dimeric inverted repeat DNA molecule that spontaneously anneals, resulting in earlier and more robust transgene expression compared with conventional single-stranded (ss) AAV genomes. Notably, scAAV can only hold a genome that is about 2.4 kb, half the size of a conventional AAV vector. In some embodiments, a dual-vector strategy may be used to overcome the small packaging capacity of AAV. For example, cis-activation, trans-splicing, overlapping, and hybrid systems may be used.

The term “AAV transfer cassette” refers to a nucleic acid comprising a transgene flanked by a first and a second ITR sequence. An AAV transfer cassette is packaged into an AAV vector during AAV vector production.

The terms “viral production cell”, “viral production cell line,” or “viral producer cell” refer to cells used to produce viral vectors. HEK293 and 239T cells are common viral production cell lines. Table 2, below, lists exemplary viral production cell lines for various viral vectors.

TABLE 2
Exemplary viral production cell lines

| | Exemplary Viral Production |
|---|---|
| Virus Vector | Cell Line(s) |
| Adenovirus | HEK293, 911, pTG6559, PER.C6, |
| | GH329, N52.E6, HeLa-E1, UR, VLI-293 |
| Adeno- | HEK293, Sf9, Se301, SeIZD2109, |
| Associated | SeUCR1, Sf9, Sf900+, Sf21 BTI-TN- |
| Virus (AAV) | 5B1-4, MG-1, Tn368, HzAm1, Ha2302, |
| | Hz2E5, High Five |
| Retrovirus | HEK293 |
| Lentivirus | 293T |

“HEK293” refers to a cell line originally derived from human embryonic kidney cells grown in tissue culture. The HEK293 cell line grows readily in culture, and is commonly used for viral production. As used herein, “HEK293” may also refer to one or more variant HEK293 cell lines, i.e., cell lines derived from the original HEK293 cell line that additionally comprise one or more genetic alterations. Many variant HEK293 lines have been developed and optimized for one or more particular applications. For example, the 293T cell line contains the SV40 large T-antigen that allows for episomal replication of transfected plasmids containing the SV40 origin of replication, leading to increased expression of desired gene products.

“Sf9” refers to an insect cell line that is a clonal isolate derived from the parental Spodoptera frugiperda cell line IPLB-Sf-21-AE. Sf9 cells can be grown in the absence of serum and can be cultured attached or in suspension.

A “transfection reagent” means a composition that enhances the transfer of nucleic acid into cells. Some transfection reagents commonly used in the art include one or more lipids that bind to nucleic acids and to the cell surface (e.g., Lipofectamine™)

### Inverted Terminal Repeat

Inverted Terminal Repeat or ITR sequences are the minimum sequences required for AAV proviral integration and for packaging of AAV DNA into virions. ITRs are involved in a variety of activities in the AAV life cycle. For example, the ITR sequences play roles in excision from the plasmid after transfection, replication of the vector genome and integration and rescue from a host cell genome.

The nucleic acids of the disclosure may comprise a 5′ ITR and/or a 3′ ITR. The ITR sequences may be about 110 to about 160 nucleotides in length, for example 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159 or 160 nucleotides. In some embodiments, the 5′ ITR is the same length as the 3′ ITR. In some embodiments, the 5′ ITR and the 3′ ITR have different lengths. In some embodiments, the 5′ ITR is longer than the 3′ ITR, and in other embodiments, the 3′ ITR is longer than the 5′ ITR.

The ITRs may be isolated or derived from the genome of any AAV, for example the AAVs listed in Table 1. In some embodiments, at least one of the 5′ ITR and the 3′ ITR is isolated or derived from the genome of AAV1, AAV2, AAV3, AAV4, AAV5, AAV6, AAV7, AAV8, AAV9, AAV10, AAV11, AAV12, AAVrh8, AAVrh10, AAVrh32.33, AAVrh74, Avian AAV or Bovine AAV. In some embodiments, at least one of the 5′ ITR and the 3′ITR may be a wildtype or mutated ITR isolated derived from a member of another parvovirus species besides AAV. For example, in some embodiments, an ITR may be a wildtype or mutant ITR isolated or derived from bocavirus or parvovirus B19.

In some embodiments, the ITR comprises a modification to promote production of a self-complementary AAV (scAAV). In some embodiments, the modification to promote production of a scAAV is deletion of the terminal resolution sequence (TRS) from the ITR. In some embodiments, the 5′ ITR is a wildtype ITR, and the 3′ ITR is a mutated ITR lacking the terminal resolution sequence. In some embodiments, the 3′ ITR is a wildtype ITR, and the 5′ ITR is a mutated ITR lacking the terminal resolution sequence. In some embodiments, the terminal resolution sequence is absent from both the 5′ ITR and the 3′ITR. In other embodiments, the modification to promote production of a scAAV is replacement of an ITR with a different hairpin-forming sequence, such as a shRNA-forming sequence.

In some embodiments, the 5′ ITR or the 3′ ITR may comprise the sequence of SEQ ID NO: 1, or a sequence at least 95%, at least 96%, at least 97%, at least 98%, or at least 99% identical thereto. In some embodiments, the 5′ ITR or the 3′ ITR may comprise the sequence of SEQ ID NO: 2, or a sequence at least 95%, at least 96%, at least 97%, at least 98%, or at least 99% identical thereto. In some embodiments, the 5′ ITR or the 3′ ITR may comprise the sequence of SEQ ID NO: 3, or a sequence at least 95%, at least 96%, at least 97%, at least 98%, or at least 99% identical thereto. In some embodiments, the 5′ ITR comprises the sequence of SEQ ID NO: 1, and the 3′ ITR comprises the sequence of SEQ ID NO: 2. In some embodiments, the 5′ ITR comprises the sequence of SEQ ID NO: 1, and the 3′ ITR comprises the sequence of SEQ ID NO: 3.

In some embodiments, the nucleic acid may comprise one or more “surrogate” ITRs, i.e., non-ITR sequences that serve the same function as ITRs. See, e.g., Xie, J. et al., Mol. Ther., 25(6): 1363-1374 (2017). In some embodiments, an ITR is replaced by a surrogate ITR. In some embodiments, the surrogate ITR comprises a hairpin-forming sequence. In some embodiments, the surrogate ITR is a short hairpin (sh)RNA-forming sequence.

### Promoters, Enhancers, Repressors and Other Regulatory Sequences

Gene expression may be controlled by nucleotide sequences such as promoters, enhancers, and/or repressors operably linked with the gene. The term “operably linked” refers to a functional linkage between a nucleic acid expression control sequence (such as a promoter, or array of transcription factor binding sites) and a second nucleic acid sequence, wherein the expression control sequence directs transcription of the nucleic acid corresponding to the second sequence.

In some embodiments, the nucleic acids or AAV transfer cassettes described herein comprise a promoter. They promoter may be, for example, a constitutive promoter or an inducible promoter. In some embodiments, the promoter is a tissue-specific promoter. As used herein, the term “promoter” refers to one or more nucleic acid control sequences that direct transcription of an operably linked nucleic acid. Promoters may include nucleic acid sequences near the start site of transcription, such as a TATA element. Promoters may also include cis-acting polynucleotide sequences that can be bound by transcription factors. A “constitutive” promoter is a promoter that is active under most environmental and developmental conditions. An “inducible” promoter is a promoter that is active under environmental or developmental regulation.

Exemplary promoters that may be used in the nucleic acids and cassettes described herein include a CMV promoter, a SV40 promoter (e.g., a SV40 early or late promoter), a metallothionein promoter, a murine mammary tumor virus (MMTV) promoter, a Rous sarcoma virus (RSV) promoter, a polyhedrin promoter, a chicken β-actin (CBA) promoter, an EF-1 alpha promoter, a dihydrofolate reductase (DHFR) promoter, a GUSB240 promoter (e.g., a human GUSB240 (hGUSB240) promoter), GUSB379 promoter (e.g., a human GUSB379 (hGUSB379) promoter), and a phosphoglycerol kinase (PGK) promoter (e.g., a human PGK (hPGK) promoter). In some embodiments, the EF-1 alpha is selected from an EF-1 alpha wildtype promoter, an EF-1 alpha short promoter, and an EF-1 alpha core promoter. In some embodiments, the promoter is selected from the group consisting of a chicken β-actin (CBA) promoter, an EF-1 alpha short promoter, an EF-1 alpha wildtype promoter, an EF-1 alpha core promoter, a hPGK promoter, a hGUSB240 promoter, and a hGUSB379 promoter. In some embodiments, the promoter comprises a sequence of any one of SEQ ID NO: 6-12, or a sequence at least 90%, at least 95%, at least 96%, at least 97%, at least 98%, or at least 99% identical thereto.

A non-limiting list of exemplary tissue-specific promoters and enhancers that may be used in the nucleic acids and cassettes described herein includes: HMG-COA reductase promoter; sterol regulatory element 1 (SRE-1); phosphoenol pyruvate carboxy kinase (PEPCK) promoter; human C-reactive protein (CRP) promoter; human glucokinase promoter; cholesterol 7-alpha hydroylase (CYP-7) promoter; beta-galactosidase alpha-2,6 sialyltransferase promoter; insulin-like growth factor binding protein (IGFBP-1) promoter; aldolase B promoter; human transferrin promoter; collagen type I promoter; prostatic acid phosphatase (PAP) promoter; prostatic secretory protein of 94 (PSP 94) promoter; prostate specific antigen complex promoter; human glandular kallikrein gene promoter (hgt-1); the myocyte-specific enhancer binding factor MEF-2; mucle creatine kinase promoter; pancreatitis associated protein promoter (PAP); elastase 1 transcriptional enhancer; pancreas specific amylase and elastase enhancer promoter; pancreatic cholesterol esterase gene promoter; uteroglobin promoter; cholesterol side-chain cleavage (SCC) promoter; gamma-gamma enolase (neuron-specific enolase, NSE) promoter; neurofilament heavy chain (NF-H) promoter; human CGL-1/granzyme B promoter; the terminal deoxy transferase (TdT), lambda 5, VpreB, and Ick (lymphocyte specific tyrosine protein kinase p561ck) promoter; the human CD2 promoter and its 3′ transcriptional enhancer; the human NK and T cell specific activation (NKGS) promoter; pp60c-src tyrosine kinase promoter; organ-specific neoantigens (OSNs), mw 40 kDa (p40) promoter; colon specific antigen-P promoter; human alpha-lactalbumin promoter; phosphoeholpyruvate carboxykinase (PEPCK) promoter, HER2/neu promoter, casein promoter, IgG promoter, Chorionic Embryonic Antigen promoter, elastase promoter, porphobilinogen deaminase promoter, insulin promoter, growth hormone factor promoter, tyrosine hydroxylase promoter, albumin promoter, alphafetoprotein promoter, acetyl-choline receptor promoter, alcohol dehydrogenase promoter, alpha or beta globin promoter, T-cell receptor promoter, the osteocalcin promoter the IL-2 promoter, IL-2 receptor promoter, whey (wap) promoter, and the MHC Class II promoter.

Gene expression may also be controlled by one or more distal “enhancer” or “repressor” elements, which can be located as much as several thousand base pairs from the start site of transcription. Enhancer or repressor elements regulate transcription in an analogous manner to cis-acting elements near the start site of transcription, with the exception that enhancer elements can act from a distance from the start site of transcription.

In some embodiments, the nucleic acids or AAV transfer cassettes described herein comprise an enhancer. The enhancer may be operably linked to a promoter. The enhancer may be, for example, a CMV enhancer. In some embodiments, the enhancer comprises the sequence of SEQ ID NO: 4 or 5, or a sequence at least 90%, at least 95%, at least 96%, at least 97%, at least 98%, or at least 99% identical thereto.

### Transgene

The nucleic acids and AAV transfer cassettes described herein may comprise a transgene sequence for expression in a target cell.

The transgene may be any heterologous nucleic acid sequence(s) of interest. Nucleic acids of interest may encode polypeptides, including therapeutic (e.g., for medical or veterinary uses) or immunogenic (e.g., for vaccines) polypeptides or RNAs. In some embodiments, the transgene is a cDNA sequence.

In some embodiments, the transgene encodes a therapeutic polypeptide. Therapeutic polypeptides include, but are not limited to, cystic fibrosis transmembrane regulator protein (CFTR), dystrophin (including mini- and micro-dystrophins, see, e.g., Vincent et al, (1993) Nature Genetics 5: 130; U.S. Patent Publication No. 2003/017131; International publication WO/2008/088895, Wang et al., Proc. Natl. Acad. Sci. USA 97: 1 3714-13719 (2000); and Gregorevic et al., Mol. Ther. 16:657-64 (2008)), myostatin propeptide, follistatin, activin type 11 soluble receptor, IGF-1, anti-inflammatory polypeptides such as the Ikappa B dominant mutant, sarcospan, utrophin (Tinsley et al, (1996) Nature 384:349), mini-utrophin, clotting factors (e.g., Factor VIII, Factor IX, Factor X, etc.), erythropoietin, angiostatin, endostatin, catalase, tyrosine hydroxylase, superoxide dismutase, leptin, the LDL receptor, lipoprotein lipase, ornithine transcarbamylase, β-globin, α-globin, spectrin, alpha-1-antitrypsin, adenosine deaminase, hypoxanthine guanine phosphoribosyl transferase, β-glucocerebrosidase, sphingomyelinase, lysosomal hexosaminidase A, branched-chain keto acid dehydrogenase, RP65 protein, cytokines (e.g., alpha-interferon, beta-interferon, gamma-interferon, interleukin-2, interleukin-4, granulocyte-macrophage colony stimulating factor, lymphotoxin, and the like), peptide growth factors, neurotrophic factors and hormones (e.g., somatotropin, insulin, insulin-like growth factors 1 and 2, platelet derived growth factor, epidermal growth factor, fibroblast growth factor, nerve growth factor, neurotrophic factor-3 and -4, brain-derived neurotrophic factor, bone morphogenic proteins [including RANKL and VEGF], glial derived growth factor, transforming growth factor-α and -β, and the like), lysosomal acid alpha-glucosidase, alpha-galactosidase A, receptors (e.g., the tumor necrosis growth factor soluble receptor), S100A1, parvalbumin, adenylyl cyclase type 6, a molecule that modulates calcium handling (e.g., SERCA_{2A}, Inhibitor 1 of PP1 and fragments thereof [e.g., WO 2006/029319 and WO 2007/100465]), a molecule that effects G-protein coupled receptor kinase type 2 knockdown such as a truncated constitutively active bARKct, anti-inflammatory factors such as RAP, anti-myostatin proteins, aspartoacylase, monoclonal antibodies (including single chain monoclonal antibodies; an exemplary Mab is the Herceptin® Mab), neuropeptides and fragments thereof (e.g., galanin, Neuropeptide Y (see, U.S. Pat. No. 7,071,172)), angiogenesis inhibitors such as Vasohibins and other VEGF inhibitors (e.g., Vasohibin 2 [see, WO JP2006/073052]). Other illustrative therapeutic polypeptides include suicide gene products (e.g., thymidine kinase, cytosine deaminase, diphtheria toxin, and tumor necrosis factor), proteins that enhance or inhibit transcription of host factors (e.g., nuclease-dead Cas9 linked to a transcription enhancer or inhibitor element, zinc-finger proteins linked to a transcription enhancer or inhibitor element, transcription activator-like (TAL) effectors linked to a transcription enhancer or inhibitor element), proteins conferring resistance to a drug used in cancer therapy, tumor suppressor gene products (e.g., p53, Rb, Wt-1), TRAIL, frataxin (FXN), FAS-ligand, and any other polypeptide that has a therapeutic effect in a subject in need thereof. A transgene may also be a monoclonal antibody or antibody fragment, for example, an antibody or antibody fragment directed against myostatin (see, e.g., Fang et al., Nature Biotechnology 23:584-590 (2005)). Therapeutic polypeptides also include those encoding reporter polypeptides (e.g., an enzyme). Reporter polypeptides are known in the art and include, but are not limited to, Green Fluorescent Protein, β-galactosidase, alkaline phosphatase, luciferase, and chloramphenicol acetyltransferase gene.

Optionally, the transgene encodes a secreted polypeptide (e.g., a polypeptide that is a secreted polypeptide in its native state or that has been engineered to be secreted, for example, by operable association with a secretory signal sequence as is known in the art).

Alternatively, in some embodiments, the transgene may encode an antisense nucleic acid, a ribozyme (e.g., as described in U.S. Pat. No. 5,877,022), RNAs that effect spliceosome-mediated/ram-splicing (see, Puttaraju et al, (1999) Nature Biotech. 17:246; U.S. Pat. Nos. 6,013,487; 6,083,702), interfering RNAs (RNAi) including siRNA, shRNA or miRNA that mediate gene silencing (see, Sharp et al, (2000) Science 287:2431), and other non-translated RNAs, such as “guide” RNAs, and the like. Exemplary untranslated RNAs include RNAi against a multiple drug resistance (MDR) gene product (e.g., to treat and/or prevent tumors and/or for administration to the heart to prevent damage by chemotherapy), RNAi against myostatin (e.g., for Duchenne muscular dystrophy), RNAi against VEGF (e.g., to treat and/or prevent tumors), RNAi against phospholamban (e.g., to treat cardiovascular disease, see, e.g., Andino et al., J. Gene Med. 10: 132-142 (2008) and Li et al., Acta Pharmacol Sin. 26:51-55 (2005)); phospholamban inhibitory or dominant-negative molecules such as phospholamban S 16E (e.g., to treat cardiovascular disease, see, e.g., Hoshijima et al. Nat. Med. 8:864-871 (2002)), RNAi to adenosine kinase (e.g., for epilepsy), and RNAi directed against pathogenic organisms and viruses (e.g., hepatitis B and/or C virus, human immunodeficiency virus, CMV, herpes simplex virus, human papilloma virus, etc.)

Further, the transgene sequence may direct alternative splicing. To illustrate, an antisense sequence (or other inhibitory sequence) complementary to the 5′ and/or 3′ splice site of dystrophin exon 51 can be delivered in conjunction with a U1 or U7 small nuclear (sn) RNA promoter to induce skipping of this exon. For example, a DNA sequence comprising a U1 or U7 snRNA promoter located 5′ to the antisense/inhibitory sequence(s) can be packaged in a cassette and delivered in an AAV vector of the disclosure.

In some embodiments, the transgene may direct gene editing. For example, the transgene may encode a gene-editing molecule such as a guide RNA or a nuclease. In some embodiments, the transgene may encode a zinc-finger nuclease, a homing endonuclease, a TALEN (transcription activator-like effector nuclease), a NgAgo (agronaute endonuclease), a SGN (structure-guided endonuclease), or a RGN (RNA-guided nuclease) such as a Cas9 nuclease or a Cpf1 nuclease.

The transgene may share homology with and recombine with a locus on a host chromosome. This approach can be utilized, for example, to correct a genetic defect in the host cell.

The transgene may be an immunogenic polypeptide, e.g., for vaccination. The transgene may encode any immunogen of interest known in the art including, but not limited to, immunogens from human immunodeficiency virus (HIV), simian immunodeficiency virus (SIV), influenza virus, HIV or SIV gag proteins, tumor antigens, cancer antigens, bacterial antigens, viral antigens, and the like.

The virus vectors according to the present disclosure provide a means for delivering transgenes into a broad range of cells, including dividing and non-dividing cells. The virus vectors can be employed to deliver a transgene to a cell in vitro, e.g., to produce a polypeptide in vitro or for ex vivo gene therapy. The virus vectors are additionally useful in a method of delivering a transgene to a subject in need thereof e.g., to express an immunogenic or therapeutic polypeptide or a functional RNA. In this manner, the polypeptide or functional RNA can be produced in vivo in the subject. The subject can be in need of the polypeptide because the subject has a deficiency of the polypeptide. Further, the method can be practiced because the production of the polypeptide or functional RNA in the subject may impart some beneficial effect.

The virus vectors can also be used to produce a polypeptide of interest or functional RNA in cultured cells or in a subject (e.g., using the subject as a bioreactor to produce the polypeptide or to observe the effects of the functional RNA on the subject, for example, in connection with screening methods).

In general, the nucleic acids and virus vectors of the present disclosure can be employed to deliver a transgene encoding a polypeptide or functional RNA to treat and/or prevent any disease state for which it is beneficial to deliver a therapeutic polypeptide or functional RNA. Illustrative disease states include, but are not limited to: cystic fibrosis (cystic fibrosis transmembrane regulator protein) and other diseases of the lung, hemophilia A (Factor VIII), hemophilia B (Factor IX), thalassemia (β-globin), anemia (erythropoietin) and other blood disorders. Alzheimer's disease (GDF; neprilysin), multiple sclerosis (β-interferon), Parkinson's disease (glial-cell line derived neurotrophic factor [GDNF]), Huntington's disease (RNAi to remove repeats), amyotrophic lateral sclerosis, epilepsy (galanin, neurotrophic factors), and other neurological disorders, cancer (endostatin, angiostatin, TRAIL, FAS-ligand, cytokines including interferons; RNAi including RNAi against VEGF or the multiple drug resistance gene product, mir-26a [e.g., for hepatocellular carcinoma]), diabetes mellitus (insulin), muscular dystrophies including Duchenne (dystrophin, mini-dystrophin, insulin-like growth factor I, a sarcoglycan [e.g., α, β, γ], RNAi against myostatic myostatin propeptide, follistatin, activin type II soluble receptor, anti-inflammatory polypeptides such as the Ikappa B dominant mutant, sarcospan, utrophin, mini-utrophin, antisense or RNAi against splice junctions in the dystrophin gene to induce exon skipping [see, e.g., WO/2003/095647], antisense against U7 snRNAs to induce exon skipping [see, e.g., WO/2006/021724], and antibodies or antibody fragments against myostatin or myostatin propeptide) and Becker, Gaucher disease (glucocerebrosidase), Hurler's disease (α-L-iduronidase), adenosine deaminase deficiency (adenosine deaminase), glycogen storage diseases (e.g., Fabry disease [α-galactosidase] and Pompe disease [lysosomal acid alpha-glucosidase]) and other metabolic disorders, congenital emphysema (alpha-1-antitrypsin), Lesch-Nyhan Syndrome (hypoxan thine guanine phosphoribosyl transferase), Niemann-Pick disease (sphingomyelinase), Tay-Sachs disease (lysosomal hexosaminidase A), Maple Syrup Urine Disease (branched-chain keto acid dehydrogenase), retinal degenerative diseases (and other diseases of the eye and retina; e.g., PDGF for macular degeneration and/or vasohibin or other inhibitors of VEGF or other angiogenesis inhibitors to treat/prevent retinal disorders, e.g., in Type I diabetes), diseases of solid organs such as brain (including Parkinson's Disease [GDNF], astrocytomas [endostatin, angiostatin and/or RNAi against VEGF], glioblastomas [endostatin, angiostatin and/or RNAi against VEGF]), liver, kidney, heart including congestive heart failure or peripheral artery disease (PAD) (e.g., by delivering protein phosphatase inhibitor I (1-1) and fragments thereof (e.g., IIC), serca2a, zinc finger proteins that regulate the phospholamban gene, Barkct, [32-adrenergic receptor, 2-adrenergic receptor kinase (BARK), phosphoinositide-3 kinase (PI3 kinase), S100A1, parvalbumin, adenylyl cyclase type 6, a molecule that effects G-protein coupled receptor kinase type 2 knockdown such as a truncated constitutively active bARKct; calsarcin, RNAi against phospholamban; phospholamban inhibitory or dominant-negative molecules such as phospholamban S16E, etc.), arthritis (insulin-like growth factors), joint disorders (insulin-like growth factor 1 and/or 2), intimal hyperplasia (e.g., by delivering enos, inos), improve survival of heart transplants (superoxide dismutase), AIDS (soluble CD4), muscle wasting (insulin-like growth factor I), kidney deficiency (erythropoietin), anemia (erythropoietin), arthritis (anti-inflammatory factors such as I RAP and TNFa soluble receptor), hepatitis (α-interferon), LDL receptor deficiency (LDL receptor), hyperammonemia (ornithine transcarbamylase), Krabbe's disease (galactocerebrosidase), Batten's disease, Friedreich's ataxia (FRDA), spinal cerebral ataxias including SCA1, SCA2 and SCA3, phenylketonuria (phenylalanine hydroxylase), autoimmune diseases, and the like. The disclosure can further be used following organ transplantation to increase the success of the transplant and/or to reduce the negative side effects of organ transplantation or adjunct therapies (e.g., by administering immunosuppressant agents or inhibitory nucleic acids to block cytokine production). As another example, bone morphogenic proteins (including BNP 2, 7, etc., RANKL and/or VEGF) can be administered with a bone allograft, for example, following a break or surgical removal in a cancer patient.

In some embodiments, the virus vectors of the present disclosure can be employed to deliver a transgene encoding a polypeptide or functional RNA to treat and/or prevent a liver disease or disorder. The liver disease or disorder may be, for example, primary biliary cirrhosis, nonalcoholic fatty liver disease (NAFLD), non-alcoholic steatohepatitis (NASH), autoimmune hepatitis, hepatitis B, hepatitis C, alcoholic liver disease, fibrosis, jaundice, primary sclerosing cholangitis (PSC), Budd-Chiari syndrome, hemochromatosis, Wilson's disease, alcoholic fibrosis, non-alcoholic fibrosis, liver steatosis, Gilbert's syndrome, biliary atresia, alpha-1-antitrypsin deficiency, alagille syndrome, progressive familial intrahepatic cholestasis, Hemophilia B, Hereditary Angioedema (HAE), Homozygous Familial Hypercholesterolemia (HoFH), Heterozygous Familial Hypercholesterolemia (HeFH), Von Gierke's Disease (GSD I), Hemophilia A, Methylmalonic Acidemia, Propionic Acidemia, Homocystinuria, Phenylketonuria (PKU), Tyrosinemia Type 1, Arginase 1 Deficiency, Argininosuccinate Lyase Deficiency, Carbamoyl-phosphate synthetase 1 deficiency, Citrullinemia Type 1, Citrin Deficiency, Crigler-Najjar Syndrome Type 1, Cystinosis, Fabry Disease, Glycogen Storage Disease 1 b, LPL Deficiency, N-Acetylglutamate Synthetase Deficiency, Ornithine Transcarbamylase Deficiency, Ornithine Translocase Deficiency, Primary Hyperoxaluria Type 1, or ADA SCID.

The virus vectors of the present disclosure can be employed to deliver a transgene used to produce induced pluripotent stem cells (iPS). For example, a virus vector of the disclosure can be used to deliver stem cell associated nucleic acid(s) into a non-pluripotent cell, such as adult fibroblasts, skin cells, liver cells, renal cells, adipose cells, cardiac cells, neural cells, epithelial cells, endothelial cells, and the like. Transgenes encoding factors associated with stem cells are known in the art. Nonlimiting examples of such factors associated with stem cells and pluripotency include Oct-3/4, the SOX family (e.g., SOX 1, SOX2, SOX3 and/or SOX 15), the Klf family (e.g., Klfl, KHZ Klf4 and/or Klf5), the Myc family (e.g., C-myc, L-myc and/or N-myc), NANOG and/or LIN28.

The virus vectors of the present disclosure can be employed to deliver a transgene to treat and/or prevent a metabolic disorder such as diabetes (e.g., insulin), hemophilia (e.g., Factor IX or Factor VIII), a lysosomal storage disorder such as a mucopolysaccharidosis disorder (e.g., Sly syndrome [β-glucuronidase], Hurler Syndrome [alpha-L-iduronidase], Scheie Syndrome [alpha-L-iduronidase], Hurler-Scheie Syndrome [alpha-L-iduronidase], Hunter's Syndrome [iduronate sulfatase], Sanfilippo Syndrome A [heparan sulfam idase], B [N-acetylglucosam inidase], C [acetyl-CoA:alpha-glucosaminide acetyltransferase], D [N-acetylglucosamine 6-sulfatase], Morquio Syndrome A [galactoses-sulfate sulfatase], B [β-galactosidase], Maroteaux-Lamy Syndrome [N-acetylgalactosamine-4-sulfatase], etc.), Fabry disease (alpha-galactosidase), Gaucher's disease (glucocerebrosidase), or a glycogen storage disorder (e.g., Pompe disease; lysosomal acid alpha-glucosidase).

In some embodiments, the transgene is useful for treating Friedreich's ataxia. In some embodiments, the transgene encodes the frataxin (FXN) protein. The frataxin protein may be, for example, the human frataxin protein. An exemplary human frataxin protein sequence is provided below (SEQ ID NO: 65):

MWTLGRRAVAGLLASPSPAQAQTLTRVPRPAELAPLCGRRGLRTDIDATC
TPRRASSNQRGLNQIWNVKKQSVYLMNLRKSGTLGHPGSLDETTYERLAE
ETLDSLAEFFEDLADKPYTFEDYDVSFGSGVLTVKLGGDLGTYVINKQTP
NKQIWLSSPSSGPKRYDWTGKNWVYSHDGVSLHELLAAELTKALKTKLDL
SSLAYSGKDA

See also Uniprot Accession No. Q16595, incorporated by reference in its entirety. In some embodiments, the frataxin protein has a sequence that is at least 90% identical, at least 95% identical, at least 96% identical, at least 97% identical, at least 98% identical, or at least 99% identical to the sequence of the human frataxin protein. In some embodiments, the frataxin protein has a sequence that is at least 90% identical, at least 95% identical, at least 96% identical, at least 97% identical, at least 98% identical, or at least 99% identical to the sequence of SEQ ID NO: 65. In some embodiments, the human frataxin protein is an isoform, variant (e.g. an alternative splice variant) or mutant form of frataxin. In some embodiments, the mutant frataxin has one or more of the substitutions shown in Table 3.

TABLE 3
Exemplary Frataxin
Amino Acid Substitutions

| Position of | |
|---|---|
| Substitution | |
| (amino acid | |
| numbering | |
| based on | |
| SEQ ID | |
| NO: 65) | Mutation |
| 106 | L → S |
| 122 | D → Y |
| 130 | G → V |
| 154 | I → F |
| 155 | W → R |
| 165 | R → C |
| 182 | L → F |
| 198 | L → R |
| 202 | W → S |
| 39-40 | RR → GG |
| 53-54 | RR → GG |
| 78-79 | LR → GG |
| 79-80 | RK → GG |

In some embodiments, the transgene comprises a frataxin cDNA that is codon optimized relative to a wildtype sequence. For example, the cDNA may be modified to remove cryptic splice acceptor/donor sites, reduce the usage of rare codons, remove ribosomal entry sites, etc. In some embodiments, the transgene comprises a frataxin cDNA that is CpG optimized. For example, the cDNA may be modified to reduce the number of CpG dinucleotides.

In some embodiments, the transgene comprises a frataxin cDNA comprising the sequence of SEQ ID NO: 19, or a sequence at least 90% identical, at least 95% identical, at least 96% identical, at least 97% identical, at least 98% identical, or at least 99% identical thereto. In some embodiments, the transgene comprises a frataxin cDNA comprising the sequence of SEQ ID NO: 20, or a sequence at least 90% identical, at least 95% identical, at least 96% identical, at least 97% identical, at least 98% identical, or at least 99% identical thereto.

### Polyadenylation (PolyA) Signal

Polyadenylation signals are nucleotide sequences found in nearly all mammalian genes and control the addition of a string of approximately 200 adenosine residues (the poly(A) tail) to the 3′ end of the gene transcript. The poly(A) tail contributes to mRNA stability, and mRNAs lacking the poly(A) tail are rapidly degraded. There is also evidence that the presence of the poly(A) tail positively contributes to the translatability of mRNA by affecting the initiation of translation.

In some embodiments, the nucleic acids and AAV transfer cassettes of the disclosure comprise one or more polyadenylation signals. In some embodiments, the nucleic acids and AAV transfer cassettes comprise two, three, four, or more polyadenylation signals. The polyadenylation signal may be the polyadenylation signal of simian virus 40 (SV40), α-globin (e.g., human α-globin, mouse α-globin, or rabbit α-globin), β-globin (e.g., human β-globin, mouse β-globin, or rabbit β-globin), human collagen, polyoma virus, human growth hormone (hGH) or bovine growth hormone (bGH), or a variant thereof.

In some embodiments, the polyadenylation signal is the bovine growth hormone (bGH) polyadenylation signal, for example a bGH polyadenylation signal having a sequence of SEQ ID NO: 21. In some embodiments, the polyadenylation signal is the human growth hormone (hGH) polyadenylation signal, for example a hGH polyadenylation signal having a sequence of SEQ ID NO: 22. In some embodiments, the polyadenylation signal is the human beta globin polyadenylation signal, for example a human beta globin polyadenylation signal having a sequence of SEQ ID NO: 23. In some embodiments, the polyadenylation signal is the rabbit beta globin polyadenylation signal, for example a rabbit beta globin polyadenylation signal having a sequence of SEQ ID NO: 24. In some embodiments, the polyadenylation signal comprises the sequence of any one of SEQ ID NO: 21-24, or a sequence at least 90%, at least 95%, at least 96%, at least 97%, at least 98%, or at least 99% identical thereto.

In some embodiments, the polyadenylation signal may be present in the nucleic acid or cassette in reverse orientation. In the reverse orientation, the polyadenylation signal may act as a safety factor. For example, the reverse orientation polyadenylation signal may prevent significant transcription from the promoter in the reverse direction.

In some embodiments, a nucleic acid or AAV transfer cassette comprises two polyadenylation signals, such as the polyadenylation signals of SEQ ID NOs: 21 and 22. In embodiments wherein the nucleic acid or AAV transfer cassette comprises two polyadenylation signals, one of the signals may be present in the reverse orientation.

### Stuffer Sequences

AAV vectors typically accept inserts of DNA having a defined size range which is generally about 4 kb to about 5.2 kb, or slightly more. Thus, for shorter sequences, it may be necessary to include additional nucleic acid in the insert fragment in order to achieve the required length which is acceptable for the AAV vector. The stuffer sequence may be isolated or derived from a non-coding region (e.g., an intronic region) of a known gene or nucleic acid sequence. The stuffer sequence may be for example, a sequence between 1-10, 10-20, 20-30, 30-40, 40-50, 50-60, 60-75, 75-100, 100-150, 150-200, 200-250, 250-300, 300-400, 400-500, 500-750, 750-1,000, 1,000-1,500, 1,500-2,000, 2,000-2,500, 2,500-3,000, 3,000-3,500, 3,500-4,000, 4,000-4,500, 4,500-5,000, 5,500-6,000, 6,000-7,000, 7,000-8,000, or 8,000-9,000 nucleotides in length. The stuffer sequence can be located in the nucleic acid or cassette at any desired position such that it does not prevent a function or activity.

In some embodiments, the nucleic acids or AAV transfer cassettes of the disclosure comprise a suffer sequence. In some embodiments, the suffer sequence comprises an intronic sequence, or a sequence derived therefrom. In some embodiments, the stuffer sequence is a chimeric sequence. In some embodiments, the stuffer sequence is isolated or derived from a gene such as alpha1-antitrypsin or albumin. In some embodiments, the suffer sequence is selected from the sequence of any one of SEQ ID NO: 25-27, or a sequence at least 90%, at least 95%, at least 96%, at least 97%, at least 98%, or at least 99% identical thereto.

### Intronic Sequences

In some embodiments, the nucleic acids and/or transfer cassettes of the disclosure may comprise an intronic sequence. The inclusion of an intronic sequence in the may recruit factors to a transcribed mRNA that are important for efficient nuclear export and translation. Thus, inclusion of an intronic sequence may enhance expression compared with expression in the absence of the intronic sequence.

In some embodiments, the intronic sequence is a hybrid or chimeric sequence. In some embodiments, the intronic sequence is isolated or derived from an intronic sequence of one or more of β-globin, chicken beta-actin, minute virus of mice (MVM), factor IX, SV40, and/or human IgG (heavy or light chain). In some embodiments, the intronic sequence comprises the sequence of any one of SEQ ID NO: 13-16, or a sequence at least 90%, at least 95%, at least 96%, at least 97%, at least 98%, or at least 99% identical thereto.

### Kozak Sequences

A Kozak sequence is a short sequence centered around the translational initiation site of eukaryotic mRNAs that allows for efficient initiation of translation of the mRNA. The ribosomal translation machinery recognizes the AUG initiation codon in the context of the Kozak sequence.

In some embodiments, the AAV transfer cassettes of the disclosure may comprise a Kozak sequence. The Kozak sequence may enhance translation efficiency and overall expression of the transgene. The Kozak sequence may be positioned immediately 5′ to the transgene sequence, or overlap with the transgene sequence.

A Kozak sequence in a nucleic acid or AAV transfer cassette of the disclosure may be a consensus sequence, or a modified version thereof. The Kozak sequence may comprise the sequence of any one of SEQ ID NO: 17-18 or 66-70, or a sequence at least 90%, at least 95%, at least 96%, at least 97%, at least 98%, or at least 99% identical thereto.

Nucleic acids and AAV Transfer Cassettes

In some embodiments, a nucleic acid or an adeno-associated virus (AAV) transfer cassette comprises one or more of an enhancer, a promoter, an intronic sequence, a Kozak sequence, a transgene sequence, a polyadenylation signal, and/or a suffer sequence. In some embodiments, a nucleic acid or an adeno-associated virus (AAV) transfer cassette comprises any combination of an enhancer, a promoter, an intronic sequence, a Kozak sequence, a transgene sequence, a polyadenylation signal, and/or a suffer sequence.

In some embodiments, a nucleic acid or an adeno-associated virus (AAV) transfer cassette comprises, from 5′ to 3′, a 5′ inverted terminal repeat (ITR), a promoter, a transgene sequence, a polyadenylation signal, and a 3′ ITR.

In some embodiments, a nucleic acid or an AAV transfer cassette comprises, from 5′ to 3′, a 5′ ITR, an enhancer, a promoter, a transgene sequence, a polyadenylation signal, and a 3′ ITR.

In some embodiments, a nucleic acid or an AAV transfer cassette comprises, from 5′ to 3′, a 5′ ITR, an enhancer, a promoter, an intronic sequence, a transgene sequence, a polyadenylation signal, and a 3′ ITR.

In some embodiments, a nucleic acid or an AAV transfer cassette comprises, from 5′ to 3′, a 5′ ITR, a promoter, an intronic sequence, a transgene sequence, a polyadenylation signal, and a 3′ ITR.

In some embodiments, a nucleic acid or an AAV transfer cassette comprises, from 5′ to 3′, a 5′ ITR, a polyA signal (reverse orientation), a promoter, an intronic sequence, a transgene sequence, a polyadenylation signal, a stuffer sequence, and a 3′ ITR.

In some embodiments, a nucleic acid or an AAV transfer cassette comprises, from 5′ to 3′, a 5′ ITR, a stuffer sequence, a polyadenylation signal (reverse orientation), a promoter, an intronic sequence, a transgene sequence, a polyadenylation signal, a stuffer sequence, and a 3′ ITR.

In some embodiments, a nucleic acid or an AAV transfer cassette comprises, from 5′ to 3′, a 5′ ITR, a stuffer sequence, a polyadenylation signal (reverse orientation), a promoter, a transgene sequence, a polyadenylation signal, a stuffer sequence, and a 3′ ITR.

In some embodiments, a nucleic acid or an AAV transfer cassette comprises, from 5′ to 3′, a 5′ ITR, a promoter, an intronic sequence, a transgene sequence, a polyadenylation signal, a suffer sequence, and a 3′ ITR.

In any of the above embodiments, the nucleic acid or AAV transfer cassette may further comprise a Kozak sequence. The Kozak sequence may be located immediately 5′ to the transgene sequence. The Kozak sequence may have the sequence of any one of SEQ ID NO: 17-18.

In some embodiments, the nucleic acid or AAV transfer cassette comprises, from 5′ to 3′, the elements shown in Table 4, or any subset thereof. A different exemplary nucleic acid or AAV transfer cassette is shown in each row in the table. An “x” indicates that the indicated element is included in the nucleic acid or AAV transfer cassette.

TABLE 4
Exemplary nucleic acids or AAV transfer cassettes (5′ to 3′)

| 5′ | | | | | | | | | | 3′ |
|---|---|---|---|---|---|---|---|---|---|---|
| ITR | Stuffer | polyA | Enhancer | Promoter | Intron | Kozak | Transgene | PolyA | Stuffer | ITR |
| X | | | X | X | X | X | X | X | | X |
| X | | | | X | X | X | X | X | | X |
| X | | | | X | X | X | X | X | | X |
| X | | | | X | X | X | X | X | | X |
| X | | | | X | X | X | X | X | | X |
| X | | | | X | X | X | X | X | | X |
| X | | | | X | X | X | X | X | | X |
| X | | | X | X | X | X | X | X | | X |
| X | | | | X | X | X | X | X | | X |
| X | | | | X | X | X | X | X | | X |
| X | | | | X | X | X | X | X | | X |
| X | | | | X | X | X | X | X | | X |
| X | | | | X | X | X | X | X | | X |
| X | | | | X | X | X | X | X | | X |
| X | | X | | X | X | X | X | X | X | X |
| X | X | X | | X | X | X | X | X | X | X |
| X | X | X | | X | X | X | X | X | X | X |
| X | X | X | | X | | X | X | X | X | X |
| X | X | X | | X | X | X | X | X | X | X |
| X | | X | | X | X | X | X | X | X | X |
| X | | | | X | X | X | X | X | X | X |
| X | | | | X | X | X | X | X | | X |
| X | | X | | X | X | X | X | X | | X |
| X | X | X | | X | X | X | X | X | X | X |
| X | X | X | | X | X | X | X | X | X | X |
| X | X | X | | X | X | X | X | X | X | |
| X | X | X | | X | X | X | X | X | X | X |
| X | | X | | X | X | X | X | X | X | X |
| X | | | | X | X | X | X | X | X | X |
| X | | | | X | X | X | X | X | | X |
| X | | | | X | X | X | X | X | | X |
| X | | | | X | X | X | X | X | | X |
| X | | | | X | X | X | X | X | | X |
| X | | | | X | X | X | X | X | | X |

In any of the above embodiments, the transgene sequence may encode the frataxin (FXN) protein. The transgene sequence may have the sequence of, for example, SEQ ID NO: 19 or SEQ ID NO: 20. In some embodiments, the transgene may encode a FXN protein having a sequence of SEQ ID NO: 65.

In any of the above embodiments, the 5′ ITR may have the sequence of SEQ ID NO: 1, and the 3′ ITR may have the sequence of SEQ ID NO: 2 or 3.

In any of the above embodiments, the enhancer may have the sequence of any one of SEQ ID NO: 4-5.

In any of the above embodiments, the promoter may have the sequence of any one of SEQ ID NO: 6-12.

In any of the above embodiments, the intronic sequence may have the sequence of any one of SEQ ID NO: 13-16.

In any of the above embodiments, the polyadenylation signal may comprise the sequence of any one of SEQ ID NO: 21-24.

In any of the above embodiments, the stuffer sequence may comprise the sequence of any one of SEQ ID NO: 25-27.

In some embodiments, the nucleic acid or AAV transfer cassette comprises, from 5′ to 3′, the elements and sequences shown in Table 5, or any subset thereof. A different exemplary nucleic acid or AAV transfer cassette is shown in each row of the table. The numbers provided in the table correspond to SEQ ID NOs.

TABLE 5
Exemplary nucleic acids or AAV transfer cassettes (5′ to 3′)

| 5′ | | | | | | | | | | 3′ |
|---|---|---|---|---|---|---|---|---|---|---|
| ITR | Stuffer | polyA | Enhancer | Promoter | Intron | Kozak | Transgene | PolyA | Stuffer | ITR |
| 1 | | | 5 | 6 | 13 | 17 | 19 | 21 | | 2 |
| 1 | | | | 7 | 15 | 17 | 19 | 22 | | 2 |
| 1 | | | | 7 | 14 | 17 | 19 | 21 | | 2 |
| 1 | | | | 11 | 15 | 17 | 19 | 22 | | 2 |
| 1 | | | | 11 | 14 | 17 | 19 | 21 | | 2 |
| 1 | | | | 11 | 16 | 17 | 19 | 22 | | 2 |
| 1 | | | | 12 | 16 | 17 | 19 | 22 | | 2 |
| 1 | | | 5 | 6 | 13 | 18 | 19 | 21 | | 2 |
| 1 | | | | 7 | 15 | 18 | 19 | 22 | | 2 |
| 1 | | | | 7 | 15 | 18 | 19 | 21 | | 2 |
| 1 | | | | 11 | 15 | 18 | 19 | 22 | | 2 |
| 1 | | | | 11 | 14 | 18 | 19 | 21 | | 2 |
| 1 | | | | 11 | 16 | 18 | 19 | 22 | | 2 |
| 1 | | | | 12 | 16 | 18 | 19 | 22 | | 2 |
| 1 | | 23 | | 10 | 14 | 18 | 19 | 22 + 24 | 25 | 3 |
| 1 | 26 | 23 | | 6 | 14 | 18 | 19 | 22 + 24 | 27 | 3 |
| 1 | 26 | 23 | | 7 | 14 | 18 | 19 | 22 + 24 | 27 | 3 |
| 1 | 26 | 23 | | 8 | | 18 | 19 | 22 + 24 | 27 | 3 |
| 1 | 26 | 23 | | 10 | 14 | 18 | 19 | 22 + 24 | 27 | 3 |
| 1 | | 23 | | 7 | 15 | 18 | 19 | 22 + 24 | 25 | 3 |
| 1 | | | | 9 | 14 | 18 | 20 | 22 + 24 | 25 | 2 |
| 1 | | | | 9 | 14 | 18 | 20 | 22 | | 2 |
| 1 | | 23 | | 10 | 14 | 17 | 19 | 22 + 24 | | 3 |
| 1 | 26 | 23 | | 6 | 14 | 17 | 19 | 22 + 24 | 27 | 3 |
| 1 | 26 | 23 | | 7 | 14 | 17 | 19 | 22 + 24 | 27 | 3 |
| 1 | 26 | 23 | | 8 | | 17 | 19 | 22 + 24 | 27 | 3 |
| 1 | 26 | 23 | | 10 | 14 | 17 | 19 | 22 + 24 | 27 | 3 |
| 1 | | 23 | | 7 | 15 | 17 | 19 | 22 + 24 | 25 | 3 |
| 1 | | | | 9 | 14 | 17 | 20 | 22 + 24 | 25 | 2 |
| 1 | | | | 9 | 14 | 17 | 20 | 22 | | 2 |
| 1 | | | | 7 | 15 | 17 | 20 | 22 | | 2 |
| 1 | | | | 7 | 15 | 17 | 20 | 22 | | 2 |
| 1 | | | | 7 | 15 | 18 | 19 | 22 | | 2 |
| 1 | | | | 7 | 18 | 18 | 20 | 22 | | 2 |

In some embodiments, the nucleic acid or AAV transfer cassette comprises the sequence of any one of SEQ ID NO: 28-64, or a sequence at least 90%, at least 95%, at least 96%, at least 97%, at least 98%, or at least 99% identical thereto.

The nucleic acids and AAV transfer cassettes described herein may be incorporated into a vector (e.g., a plasmid or a bacmid) using standard molecular biology techniques. The vector (e.g., plasmid or bacmid) may further comprise one or more genetic elements used during production of AAV, including, for example, AAV rep and cap genes, and helper virus protein sequences.

### Recombinant AAVs and AAV Production Methods

The nucleic acids and AAV transfer cassettes, and vectors (e.g., plasmids) comprising the nucleic acids and AAV transfer cassettes described herein, may be used to produce recombinant AAV vectors. The AAV vectors may comprise a single stranded genome or a double stranded genome (i.e., a scAAV). High titer AAV preparations can be produced using techniques known in the art, such as standard triple transfection or baculoviral production methods.

Typically, methods for production of AAV vectors include 4 components: plasmids acting in trans and the transgene acting in cis. These components include: 1) a plasmid containing the AAV Rep and Cap genes for capsid formation and replication, 2) a plasmid containing adenovirus helper genes, 3) a cassette containing the transgene enclosed by two inverted terminal repeats (ITR), and 4) a viral packaging cell line. Since AAV is highly infectious and naturally present in a large percentage of the human population, cell cultures and all materials may be thoroughly tested for transient wild type AAV infection before use.

In some embodiments, a method for producing a recombinant AAV vector comprises contacting an AAV producer cell (e.g., an HEK293 cell) with a nucleic acid, AAV transfer cassette or vector (e.g., plasmid) of the disclosure. In some embodiments, the method further comprises contacting the AAV producer cell with one or more additional vectors (e.g., plasmids) encoding, for example, AAV rep and cap genes, and helper virus protein sequences. In some embodiments, the method further comprises maintaining the AAV producer cell under conditions such that AAV is produced.

In some embodiments, a method for producing a recombinant AAV vector comprises contacting an AAV producer cell (e.g., an insect cell such as a Sf9 cell) with at least one insect cell-compatible vector comprising a nucleic acid or AAV transfer cassette of the disclosure. An “insect cell-compatible vector” is any compound or formulation, biological or chemical, which formulation facilitates transformation or transfection of an insect cell with a nucleic acid. In some embodiments, the insect cell-compatible vector is a baculoviral vector. In some embodiments, the method further comprises maintaining the insect cell under conditions such that AAV is produced.

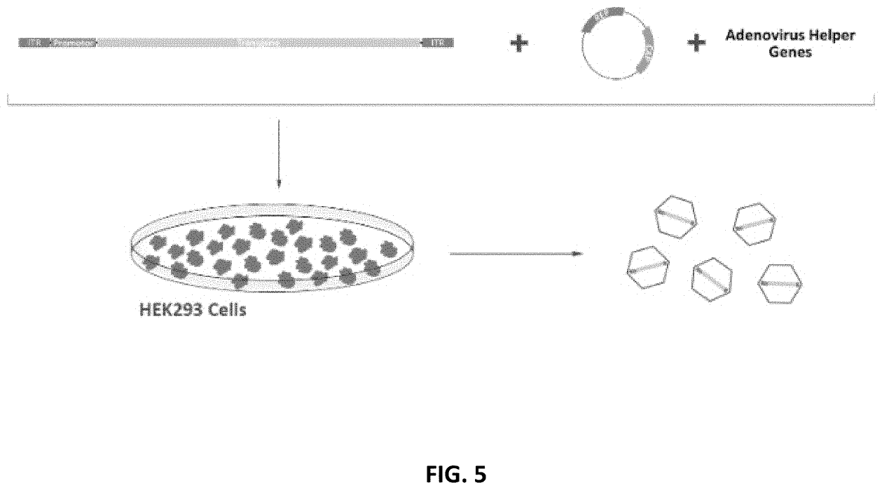

In some embodiments, an AAV producer cell is transfected (e.g., using a transfection reagent) with three plasmids: (1) a first plasmid comprising a nucleic acid or AAV transfer cassette of the disclosure, (2) a second plasmid comprising AAV rep and cap gene sequences, and (3) a third plasmid comprising helper virus protein sequences. See, e.g., FIG. 5. The AAV producer cell may be any of the cells listed in Table 2. The AAV producer cell may subsequently be maintained under conditions such that AAV is produced. The AAV may then be purified using standard techniques, such as cesium chloride (CsCl) gradient centrifugation or column chromatography techniques.

The recombinant AAV vectors produced may comprise a capsid of any serotype, for example AAV1, AAV2, AAV3, AAV4, AAV5, AAV6, AAV7, AAV8, AAV9, AAV10, AAV11, AAV12, AAVrh8, AAVrh10, AAVrh32.33, AAVrh74, Avian AAV and Bovine AAV. In some embodiments, the recombinant AAV vectors produced may comprise a capsid protein with one or more amino acid modifications (e.g., substitutions and/or deletions) compared to the native AAV capsid. For example, the recombinant AAV vectors may comprise modified AAV capsids derived from AAV1, AAV2, AAV3, AAV4, AAV5, AAV6, AAV7, AAV8, AAV9, AAV10, AAV11, AAV12, AAVrh8, AAVrh10, AAVrh32.33, AAVrh74, Avian AAV and Bovine AAV. In some embodiments, the AAV vector produced is an AAV9. In some embodiments, the AAV vector produced is an AAV1. In some embodiments, the AAV vector produced is an AAV4.

The recombinant AAV vectors may be used to transduce target cells with the transgene sequence, for example by contacting the recombinant AAV vector with a target cell.

### Compositions

Also provided are compositions comprising a nucleic acid, AAV transfer cassette, plasmid, cell, or recombinant AAV vector of the disclosure. In some embodiments, the compositions are liquid compositions. In some embodiments, the compositions are solid compositions.

In some embodiments, a pharmaceutical composition comprising a nucleic acid, AAV transfer cassette, a plasmid, a cell, or a recombinant AAV vector of the disclosure is provided. Pharmaceutical compositions according to the present disclosure, and for use in accordance with the present disclosure, may comprise, in addition to a nucleic acid, AAV transfer cassette, plasmid, cell, or recombinant AAV vector, a pharmaceutically acceptable excipient, carrier, buffer, stabilizer or other materials (e.g., diluents, adjuvants, fillers, preservatives, anti-oxidants, lubricants, solubilizers, surfactants (e.g., wetting agents), masking agents, coloring agents, flavoring agents, and sweetening agents). Such materials should preferably be non-toxic. Suitable carriers, diluents, excipients, etc. can be found in standard pharmaceutical texts. See, for example, Handbook of Pharmaceutical Additives, 2nd Edition (eds. M. Ash and I. Ash), 2001 (Synapse Information Resources, Inc., Endicott, N.Y., USA), Remington's Pharmaceutical Sciences, 20th edition, pub. Lippincott, Williams & Wilkins, 2000; and Handbook of Pharmaceutical Excipients, 2nd edition, 1994. The precise nature of the carrier or other material will depend on the route of administration, which may be oral, or by injection, e.g. cutaneous, subcutaneous, or intravenous.

Pharmaceutical compositions for oral administration may be in tablet, capsule, powder or liquid form. A tablet may comprise a solid carrier or an adjuvant. Liquid pharmaceutical compositions generally comprise a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included. A capsule may comprise a solid carrier such a gelatin.

For intravenous, cutaneous or subcutaneous injection, or injection at the site of affliction, the pharmaceutical composition may be in the form of a parenterally acceptable aqueous solution which is pyrogen-free and has suitable pH, isotonicity and stability. Suitable solutions may comprise, for example, isotonic vehicles such as Sodium Chloride, Ringer's solution, and/or Lactated Ringer's solution. Preservatives, stabilizers, buffers, antioxidants and/or other additives may be included, as required.

### Methods of Treatment

The AAV vectors of the disclosure, including AAV vectors prepared using the nucleic acids or AAV transfer cassettes of the disclosure, may be used to treat or prevent a disease, disorder, or other condition a subject in need thereof. The subject may be a mammal or an avian. In some embodiments, the mammal is a cat, dog, mouse, rat, horse, cow, pig, guinea pig, or non-human primate. In some embodiments, the subject is a human. The human may be a pediatric subject, an adult subject, or a geriatric subject.

The AAV vectors of the disclosure, or compositions comprising the same, may be contacted with a cell in vivo or ex vivo. The cell may then be maintained under conditions sufficient for expression of the transgene in the cell.

The AAV vectors of the disclosure, or compositions comprising the same, may be administered to a subject in need thereof. Administration can be by any means known in the art. Optionally, the virus vector and/or composition is delivered in a therapeutically effective dose in a pharmaceutically acceptable carrier. In some embodiments, a therapeutically effective dose of the virus vector and/or composition is delivered.

Dosages of the virus vector and/or composition to be administered to a subject depend upon the mode of administration, the disease or condition to be treated and/or prevented, the individual subject's condition, the particular virus vector or composition, the nucleic acid to be delivered, and the like, and can be determined in a routine manner. Exemplary doses for achieving therapeutic effects are titers of at least about 10⁵, at least about 10⁶, at least about 10⁷, at least about 10⁸, at least about 10⁹, at least about 10¹⁰, at least about 10¹¹, at least about 10¹², at least about 10¹³, at least about 10¹⁴, at least about 10¹⁵ transducing units, optionally about 10⁸ to about 10¹³ transducing units.

In particular embodiments, more than one administration (e.g., two, three, four or more administrations) may be employed to achieve the desired level of gene expression over a period of various intervals, e.g., daily, weekly, monthly, yearly, etc.

Exemplary modes of administration include oral, rectal, transmucosal, intranasal, inhalation (e.g., via an aerosol), buccal (e.g., sublingual), vaginal, intrathecal, intraocular, transdermal, in utero (or in ovo), parenteral (e.g., intravenous, subcutaneous, intradermal, intramuscular (including administration to skeletal, diaphragm and/or cardiac muscle), intradermal, intrapleural, intracerebral, and intraarticular), topical (e.g., to both skin and mucosal surfaces, including airway surfaces, and transdermal administration), intralymphatic, and the like, as well as direct tissue or organ injection (e.g., to liver, skeletal muscle, cardiac muscle, diaphragm muscle or brain). Administration can also be to a tumor (e.g., in or near a tumor or a lymph node). The most suitable route in any given case will depend on the nature and severity of the condition being treated and/or prevented and on the nature of the particular vector that is being used.

In some embodiments, an AAV vector or composition comprising the vector may be administered by direct injection into cardiac or central nervous system (CNS) tissue. In some embodiments, the AAV vector or composition comprising the vector, may be delivered intracranially including intrathecal, intraneural, intra-cerebral, or intra-ventricular administration. In some embodiments, the AAV vector or composition comprising the vector, may be delivered to the heart by direct administration into the myocardium by epicardiac injection followed by minithoractomy, by intracoronary injection, or by endomyocardic injection.

Delivery to a target tissue can also be achieved by delivering a depot comprising the virus vector and/or capsid. In representative embodiments, a depot comprising the virus vector and/or capsid is implanted into skeletal, cardiac and/or diaphragm muscle tissue or the tissue can be contacted with a film or other matrix comprising the virus vector and/or capsid. Such implantable matrices or substrates are described in U.S. Pat. No. 7,201,898.

Administration of an AAV may result in robust and persistent transgene expression in a target cell or tissue. For example, transgene expression may persist for at least 1 week, at least 2 weeks, at least 3 weeks, at least 4 weeks, at least 5 weeks, at least 6 weeks, at least 7 weeks, at least 8 weeks, at least 3 months, at least 4 months, at least 5 months, at least 6 months, at least 12 months, at least 24 months, at least 36 moths, or longer.

In some embodiments, a method for treating a subject in need thereof comprises administering to the subject a therapeutically effective amount of a nucleic acid, an AAV transfer cassette, a plasmid, a cell, or a recombinant AAV of the disclosure. In some embodiments, the subject is a human subject. In some embodiments, the subject suffers from Friedreich's Ataxia. The administering may result in expression of a therapeutically effective amount of FXN protein in a CNS tissue (e.g., a neuronal tissue) or a cardiac tissue of the subject.

In some embodiments, the administering may result in alleviation of one or more symptoms of Friedrich's Ataxia. For example, the administering may (1) improve coordination (ataxia) in the arms and legs of the subject, (2) increase energy levels and/or decrease fatigue and muscle loss in the subject, (3) improve vision, hearing loss, or speech in the subject, (3) decrease scoliosis or the rate of progression thereof, (4) improve the symptoms of diabetes such as insulin sensitivity, or (5) ameliorate heart conditions such as hypertrophic cardiomyopathy or arrhythmia. The improvement in the subject due to treatment may be an improvement as compared to the subject pre-treatment, or as compared to typical subjects with Friedrich's ataxia.

In some embodiments, the administering may result in an extension of the lifespan of the subject. For example, the administering may extend the lifespan of the subject by about 1 year, about 2 years, about 3 years, about 4 years, about 5 years, about 5-10 years, or greater than 10 years compared to a typical subject that has Friedrich's ataxia.

### EXAMPLES

The following examples, which are included herein for illustration purposes only, are not intended to be limiting.

### Example 1: Preparation of a Recombinant AAV Vector in Mammalian Cells

Three plasmids are provided. The first plasmid comprises a transfer cassette comprising a cDNA encoding human frataxin (SEQ ID NO: 19 or 20) flanked by two ITRs (SEQ ID NO: 1, SEQ ID NO: 2 or 3), and has the sequence of any one of SEQ ID NO: 28-64. The second plasmid comprises sequences encoding the Rep and Cap genes. The third plasmid comprises various “helper” sequences required for AAV production (E4, E2a, and VA).

The three plasmids are transfected into viral production cells (e.g., HEK293) using an appropriate transfection reagent (e.g., Lipofectamine™). After incubation at 37° C. for a predetermined period of time, AAV particles are collected from the media or the cells are lysed to release the AAV particles. The AAV particles are then purified, titered, and may be stored at −80° C. for later use.

### Example 2: Preparation of a Recombinant AAV Vector in Insect Cells

A first recombinant baculoviral vector is provided. The first recombinant baculoviral vector comprises a transfer cassette sequence comprising a cDNA encoding human frataxin (SEQ ID NO: 19 or 20) flanked by two ITRs (SEQ ID NO: 1, SEQ ID NO: 2 or 3), wherein the transfer cassette has the sequence of any one of SEQ ID NO: 28-64.

Insect cells (e.g., Sf9) are co-infected in suspension culture with the first recombinant baculoviral vector and a least one additional recombinant baculoviral vector comprising sequences encoding the AAV Rep and Cap proteins. After incubation at 28° C. for a predetermined period of time, AAV particles are collected from the media or the cells are lysed to release the AAV particles. The AAV particles are then purified, titered, and may be stored at −80° C. for later use.

### Example 3: Recombinant AAV Packaging FXN Transgene Transduces Heart Cells In Vivo and Extends Lifespan of FXN Deficient Mice

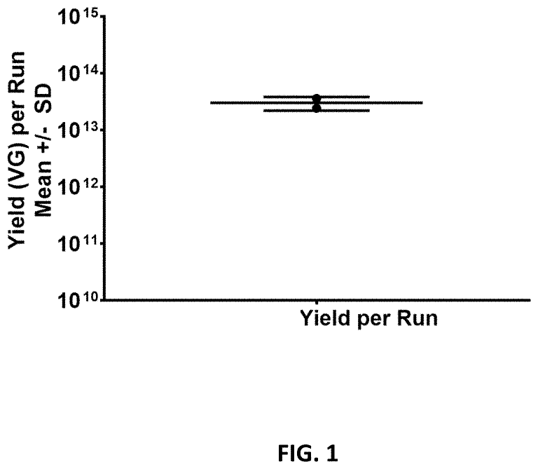

A plasmid comprising an AAV transfer cassette (SEQ ID NO: 32), including a human FXN transgene, was prepared using standard cloning techniques (FXN plasmid). A composition comprising the FXN plasmid, a second plasmid comprising sequences encoding AAV Rep and Cap (AAV9) genes, and a third plasmid comprising sequences encoding AAV helper sequences was prepared and used to transfect HEK293 cells using a standard “triple transfection” protocol. The HEK293 cells were maintained under standard culture conditions (37° C., 5% CO₂) to allow for production of recombinant, self-complementary AAV9 vectors. This procedure was repeated several times, and AAV9 vector yield was quantified using ddPCR®. As shown in FIG. 1, the yield of AAV9 packaging the FXN transgene (AAV9-FXN) in each run was between 10¹³ and 10¹⁴ vector genomes.

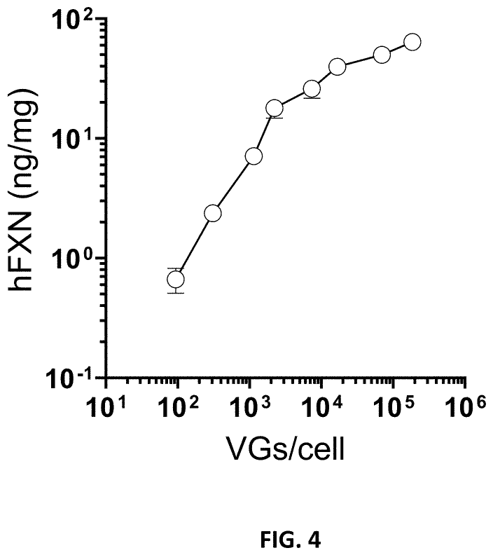

The recombinant AAV9-FXN was used to transduce Lec2 cells in culture. FIG. 4 shows expression of human FXN (ng/mg) in cultured Lec2 cells transduced with various doses of AAV9-FXN. Higher expression of hFXN was observed at higher doses of vector were used.

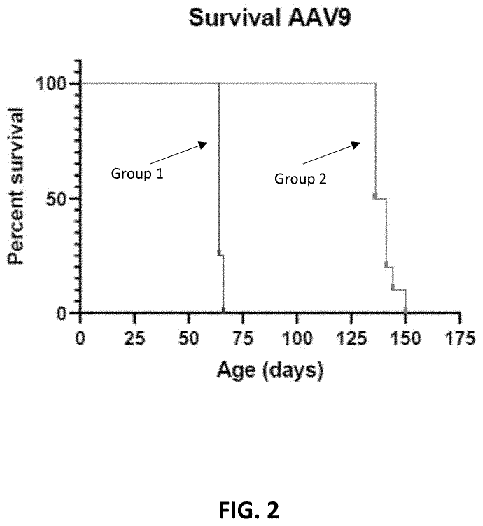

The recombinant AAV9-FXN was also used to infect mice lacking FXN in cardiac and skeletal muscle (FXN^{flox/flox}MCKCre⁺). Mice were treated with either saline or AAV9-FXN (5×10¹³ vg/kg) at 3 weeks of age, and survival was monitored. As shown in FIG. 2, treatment with AAV9-FXN significantly increased lifespan. The median survival of the saline-injected mice was 64 days, whereas the median survival of AAV9-FXN injected mice was 138.5 days.

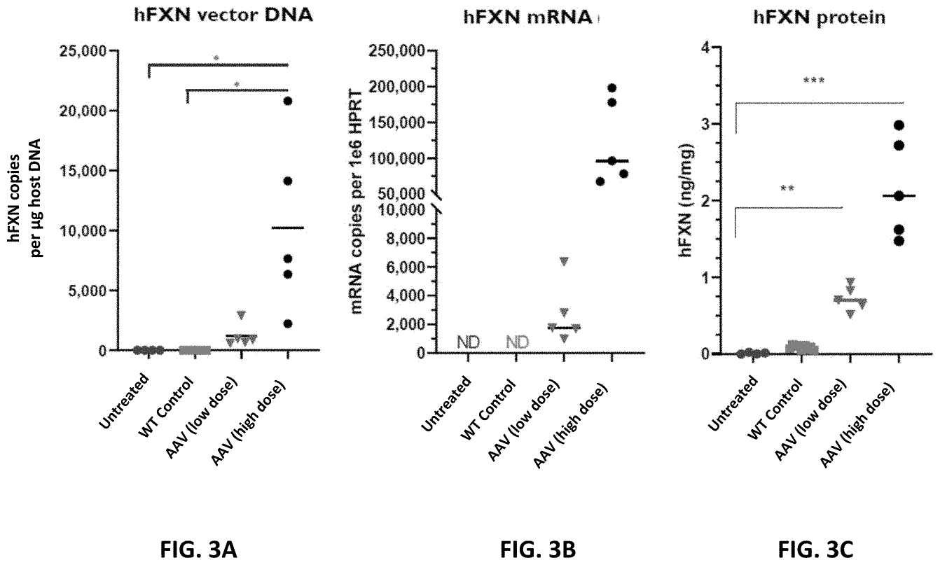

In a separate experiment, FXN deficient mice were treated with either saline, or a low or high dose of AAV9-FXN (1×10¹³ or 5×10¹³ vg/kg, respectively) at 3 weeks of age. Mice were sacrificed 3 weeks post-treatment, and heart tissue was analyzed. As shown in FIG. 3A, human FXN (hFXN) DNA was detectable in heart tissue from AAV9-FXN treated mice. The hFXN DNA was transcribed to RNA (FIG. 3B) and translated into protein (FIG. 3C). The higher dose of AAV9-FXN led to higher levels of FXN DNA, RNA, and protein in the heart samples.

Taken together, these data show that an AAV transfer cassette of the disclosure comprising the FXN transgene can be used to produce recombinant AAV vectors, and can be used to transduce the cells of a subject in vivo.

### NUMBERED EMBODIMENTS

Notwithstanding the appended claims, the disclosure sets forth the following numbered embodiments of the disclosure:

1. A nucleic acid comprising, from 5′ to 3′: a 5′ inverted terminal repeat (ITR); a promoter; a transgene sequence; a polyadenylation signal; and a 3′ ITR; wherein the transgene sequence encodes the frataxin (FXN) protein.

2. The nucleic acid of embodiment 1, wherein at least one of the 5′ ITR and the 3′ ITR is about 110 to about 160 nucleotides in length.

3. The nucleic acid of embodiment 1 or 2, wherein the 5′ ITR is the same length as the 3′ ITR.

4. The nucleic acid of embodiment 1 or 2, wherein the 5′ ITR and the 3′ ITR have different lengths.

5. The nucleic acid of any one of embodiments 1-4, wherein at least one of the 5′ ITR and the 3′ ITR is isolated or derived from the genome of AAV1, AAV2, AAV3, AAV4, AAV5, AAV6, AAV7, AAV8, AAV9, AAV10, AAV11, AAV12, AAVrh8, AAVrh10, AAVrh32.33, AAVrh74, Avian AAV or Bovine AAV.

6. The nucleic acid of embodiment 1, wherein the 5′ ITR comprises the sequence of SEQ ID NO: 1, or a sequence at least 95% identical thereto.

7. The nucleic acid of any one of embodiments 1-6, wherein the 3′ ITR comprises the sequence of SEQ ID NO: 2, or a sequence at least 95% identical thereto.

8. The nucleic acid of any one of embodiments 1-7, wherein the 3′ ITR comprises the sequence of SEQ ID NO: 3, or a sequence at least 95% identical thereto.

9. The nucleic acid of any one of embodiments 1-8, wherein the promoter drives expression of the transgene.

10. The nucleic acid of any one of embodiments 1-9, wherein the promoter is a constitutive promoter.

11. The nucleic acid of any one of embodiments 1-9, wherein the promoter is an inducible promoter.

12. The nucleic acid of any one of embodiments 1-11, wherein the promoter is a tissue-specific promoter.

13. The nucleic acid of any one of embodiments 1-12, wherein the promoter is selected from the group consisting of the CMV promoter, the SV40 early promoter, the SV40 late promoter, the metallothionein promoter, the murine mammary tumor virus (MMTV) promoter, the Rous sarcoma virus (RSV) promoter, the polyhedrin promoter, the chicken β-actin (CBA) promoter, the EF-1 alpha promoter, the EF-1 alpha short promoter, the EF-1 alpha core promoter, the dihydrofolate reductase (DHFR) promoter, the GUSB240 promoter, the GUSB379 promoter, and the phosphoglycerol kinase (PGK) promoter.

14. The nucleic acid of embodiment 13, wherein the promoter is the chicken β-actin (CBA) promoter.

15. The nucleic acid of embodiment 13, wherein the promoter is the EF-1 alpha promoter, the EF-1 alpha short promoter, or the EF-1 alpha core promoter.

16. The nucleic acid of embodiment 13, wherein the promoter is the GUSB240 promoter.

17. The nucleic acid of embodiment 13, wherein the promoter is the GUSB379 promoter.

18. The nucleic acid of embodiment 13, wherein the promoter is the PGK promoter.

19. The nucleic acid of any one of embodiments 1-12, wherein the promoter comprises a sequence selected from any one of SEQ ID NO: 6-12, or a sequence at least 95% identical thereto.

20. The nucleic acid of any one of embodiments 1-19, wherein the FXN protein is the human FXN protein.

21. The nucleic acid of any one of embodiments 1-20, wherein the FXN protein has the sequence of SEQ ID NO: 65, or a sequence that is at least 95% identical thereto.

22. The nucleic acid of any one of embodiments 1 to 21, wherein the transgene sequence is CpG optimized.

23. The nucleic acid of any one of embodiments 1-21, wherein the transgene sequence comprises SEQ ID NO: 19 or 20, or a sequence that is at least 95% identical thereto.

24. The nucleic acid of any one of embodiments 1-24, wherein the nucleic acid comprises a Kozak sequence immediately 5′ to the transgene sequence.

25. The nucleic acid of embodiment 24, wherein the Kozak sequence comprises the sequence of SEQ ID NO: 17 or 18, or a sequence at least 95% identical thereto.

26. The nucleic acid of any one of embodiments 1-25, wherein the polyadenylation signal is selected from the polyadenylation signal of simian virus 40 (SV40), human α-globin, rabbit α-globin, human β-globin, rabbit β-globin, human collagen, polyoma virus, human growth hormone (hGH) and bovine growth hormone (bGH).

27. The nucleic acid of embodiment 26, wherein the polyadenylation signal is the bovine growth hormone polyadenylation signal.

28. The nucleic acid of embodiment 26, wherein the polyadenylation signal is the human growth hormone polyadenylation signal.

29. The nucleic acid of embodiment 26, wherein the polyadenylation signal is the human β-globin polyadenylation signal.

30. The nucleic acid of embodiment 26, wherein the polyadenylation signal is the rabbit β-globin polyadenylation signal.

31. The nucleic acid of any one of embodiments 1-25, wherein the polyadenylation signal comprises the sequence of any one of SEQ ID NO: 21-24, or a sequence at least 95% identical thereto.

32. The nucleic acid of any one of embodiments 1-31, wherein the nucleic acid further comprises an enhancer.

33. The nucleic acid of embodiment 32, wherein the enhancer is a CMV enhancer.

34. The nucleic acid of embodiment 32, wherein the enhancer comprises the sequence of SEQ ID NO: 4 or 5, or a sequence at least 95% identical thereto.

35. The nucleic acid of any one of embodiments 1-34, wherein the cassette further comprises an intronic sequence.

36. The nucleic acid of embodiment 35, wherein the intronic sequence is a chimeric sequence.

37. The nucleic acid of embodiment 35, wherein the intronic sequence is a hybrid sequence.

38. The nucleic acid of embodiment 35, wherein the intronic sequence comprises sequences isolated or derived from intronic sequences of one or more of β-globin, chicken beta-actin, minute virus of mice, and human IgG.

39. The nucleic acid of embodiment 35, wherein the intronic sequence comprises the sequence of any one of SEQ ID NO: 13-16, or a sequence at least 95% identical thereto.

40. The nucleic acid of any one of embodiments 1-39, wherein the nucleic acid further comprises at least one stuffer sequence.

41. The nucleic acid of embodiment 40, wherein the nucleic acid comprises two stuffer sequences.

42. The nucleic acid of embodiment 40, wherein the at least one stuffer sequence comprises the sequence of any one of SEQ ID NO: 25-27, or a sequence at least 95% identical thereto.

43. The nucleic acid of embodiment 1, wherein the nucleic acid comprises the sequence of any one of SEQ ID NO: 28-64, or a sequence at least 95% identical thereto.

44. A plasmid comprising the nucleic acid of any one of embodiments 1-43.

45. A cell comprising the nucleic acid of any one of embodiments 1-43 or the plasmid of embodiment 44.

46. A method of producing a recombinant AAV vector, the method comprising contacting an AAV producer cell with the nucleic acid of any one of embodiments 1-43 or the plasmid of embodiment 44.

47. A recombinant AAV vector produced by the method of embodiment 46.

48. The recombinant AAV vector of embodiment 47, wherein the recombinant AAV vector is a single-stranded AAV (ssAAV).

49. The recombinant AAV vector of embodiment 47, wherein the recombinant AAV vector is a self-complementary AAV (scAAV).

50. The recombinant AAV vector of any one of embodiments 47-49, wherein the AAV vector comprises a capsid protein of AAV1, AAV2, AAV3, AAV4, AAV5, AAV6, AAV7, AAV8, AAV9, AAV10, AAV11, AAV12, AAVrh8, AAVrh10, AAVrh32.33, AAVrh74, Avian AAV or Bovine AAV.

51. The recombinant AAV vector of any one of embodiments 47-49, wherein the AAV vector comprises a capsid protein with one or more substitutions or mutations compared to a wildtype AAV capsid protein.

52. A composition comprising the nucleic acid of any one of embodiments 1-43, the plasmid of embodiment 44, the cell of embodiment 45, or the recombinant AAV vector of any one of embodiments 47-51.

53. A method for treating a subject in need thereof comprising administering to the subject a therapeutically effective amount of the nucleic acid of any one of embodiments 1-43, the plasmid of embodiment 44, the cell of embodiment 45, or the recombinant AAV vector of any one of embodiments 47-41.

54. The method of embodiment 53, wherein the subject has Friedreich's Ataxia.

55. The method of embodiment 53 or 54, wherein the subject is a human subject.

56. The method of any one of embodiments 53-55, wherein the nucleic acid, the plasmid, the cell, or the recombinant AAV vector is administered by direct injection into the central nervous system.

The foregoing is illustrative of the present invention, and is not to be construed as limiting thereof. The invention is defined by the following claims, with equivalents of the claims to be included therein.

What is claimed is:

1. A nucleic acid comprising, from 5′ to 3′:
a 5′ inverted terminal repeat (ITR);
a promoter;
a transgene sequence;
a polyadenylation signal; and
a 3′ ITR;
wherein the transgene sequence encodes the frataxin (FXN) protein.

2. The nucleic acid of claim 1, wherein at least one of the 5′ ITR and the 3′ ITR is about 110 to about 160 nucleotides in length.

3. The nucleic acid of claim 1 or 2, wherein the 5′ ITR is the same length as the 3′ ITR.

4. The nucleic acid of claim 1 or 2, wherein the 5′ ITR and the 3′ ITR have different lengths.

5. The nucleic acid of any one of claims 1-4, wherein at least one of the 5′ ITR and the 3′ ITR is isolated or derived from the genome of AAV1, AAV2, AAV3, AAV4, AAV5, AAV6, AAV7, AAV8, AAV9, AAV10, AAV11, AAV12, AAVrh8, AAVrh10, AAVrh32.33, AAVrh74, Avian AAV or Bovine AAV.

6. The nucleic acid of claim 1, wherein the 5′ ITR comprises the sequence of SEQ ID NO: 1, or a sequence at least 95% identical thereto.

7. The nucleic acid of any one of claims 1-6, wherein the 3′ ITR comprises the sequence of SEQ ID NO: 2, or a sequence at least 95% identical thereto.

8. The nucleic acid of any one of claims 1-7, wherein the 3′ ITR comprises the sequence of SEQ ID NO: 3, or a sequence at least 95% identical thereto.

9. The nucleic acid of any one of claims 1-8, wherein the promoter drives expression of the transgene.

10. The nucleic acid of any one of claims 1-9, wherein the promoter is a constitutive promoter.

11. The nucleic acid of any one of claims 1-9, wherein the promoter is an inducible promoter.

12. The nucleic acid of any one of claims 1-11, wherein the promoter is a tissue-specific promoter.

13. The nucleic acid of any one of claims 1-12, wherein the promoter is selected from the group consisting of the CMV promoter, the SV40 early promoter, the SV40 late promoter, the metallothionein promoter, the murine mammary tumor virus (MMTV) promoter, the Rous sarcoma virus (RSV) promoter, the polyhedrin promoter, the chicken β-actin (CBA) promoter, the EF-1 alpha promoter, the EF-1 alpha short promoter, the EF-1 alpha core promoter, the dihydrofolate reductase (DHFR) promoter, the GUSB240 promoter, the GUSB379 promoter, and the phosphoglycerol kinase (PGK) promoter.

14. The nucleic acid of claim 13, wherein the promoter is the chicken β-actin (CBA) promoter.

15. The nucleic acid of claim 13, wherein the promoter is the EF-1 alpha promoter, the EF-1 alpha short promoter, or the EF-1 alpha core promoter.

16. The nucleic acid of claim 13, wherein the promoter is the GUSB240 promoter.

17. The nucleic acid of claim 13, wherein the promoter is the GUSB379 promoter.

18. The nucleic acid of claim 13, wherein the promoter is the PGK promoter.

19. The nucleic acid of any one of claims 1-12, wherein the promoter comprises a sequence selected from any one of SEQ ID NO: 6-12, or a sequence at least 95% identical thereto.

20. The nucleic acid of any one of claims 1-19, wherein the FXN protein is the human FXN protein.

21. The nucleic acid of any one of claims 1-20, wherein the FXN protein has the sequence of SEQ ID NO: 65, or a sequence that is at least 95% identical thereto.

22. The nucleic acid of any one of claims 1 to 21, wherein the transgene sequence is CpG optimized.

23. The nucleic acid of any one of claims 1-21, wherein the transgene sequence comprises SEQ ID NO: 19 or 20, or a sequence that is at least 95% identical thereto.

24. The nucleic acid of any one of claims 1-24, wherein the nucleic acid comprises a Kozak sequence immediately 5′ to the transgene sequence.

25. The nucleic acid of claim 24, wherein the Kozak sequence comprises the sequence of SEQ ID NO: 17 or 18, or a sequence at least 95% identical thereto.

26. The nucleic acid of any one of claims 1-25, wherein the polyadenylation signal is selected from the polyadenylation signal of simian virus 40 (SV40), human α-globin, rabbit α-globin, human β-globin, rabbit β-globin, human collagen, polyoma virus, human growth hormone (hGH) and bovine growth hormone (bGH).

27. The nucleic acid of claim 26, wherein the polyadenylation signal is the bovine growth hormone polyadenylation signal.

28. The nucleic acid of claim 26, wherein the polyadenylation signal is the human growth hormone polyadenylation signal.

29. The nucleic acid of claim 26, wherein the polyadenylation signal is the human β-globin polyadenylation signal.

30. The nucleic acid of claim 26, wherein the polyadenylation signal is the rabbit β-globin polyadenylation signal.

31. The nucleic acid of any one of claims 1-25, wherein the polyadenylation signal comprises the sequence of any one of SEQ ID NO: 21-24, or a sequence at least 95% identical thereto.

32. The nucleic acid of any one of claims 1-31, wherein the nucleic acid further comprises an enhancer.

33. The nucleic acid of claim 32, wherein the enhancer is a CMV enhancer.

34. The nucleic acid of claim 32, wherein the enhancer comprises the sequence of SEQ ID NO: 4 or 5, or a sequence at least 95% identical thereto.

35. The nucleic acid of any one of claims 1-34, wherein the cassette further comprises an intronic sequence.

36. The nucleic acid of claim 35, wherein the intronic sequence is a chimeric sequence.

37. The nucleic acid of claim 35, wherein the intronic sequence is a hybrid sequence.

38. The nucleic acid of claim 35, wherein the intronic sequence comprises sequences isolated or derived from intronic sequences of one or more of β-globin, chicken beta-actin, minute virus of mice, and human IgG.

39. The nucleic acid of claim 35, wherein the intronic sequence comprises the sequence of any one of SEQ ID NO: 13-16, or a sequence at least 95% identical thereto.

40. The nucleic acid of any one of claims 1-39, wherein the nucleic acid further comprises at least one stuffer sequence.

41. The nucleic acid of claim 40, wherein the nucleic acid comprises two stuffer sequences.

42. The nucleic acid of claim 40, wherein the at least one stuffer sequence comprises the sequence of any one of SEQ ID NO: 25-27, or a sequence at least 95% identical thereto.

43. The nucleic acid of claim 1, wherein the nucleic acid comprises the sequence of any one of SEQ ID NO: 28-64, or a sequence at least 95% identical thereto.

44. A plasmid comprising the nucleic acid of any one of claims 1-43.

45. A cell comprising the nucleic acid of any one of claims 1-43 or the plasmid of claim 44.

46. A method of producing a recombinant AAV vector, the method comprising contacting an AAV producer cell with the nucleic acid of any one of claims 1-43 or the plasmid of claim 44.

47. A recombinant AAV vector produced by the method of claim 46.

48. The recombinant AAV vector of claim 47, wherein the recombinant AAV vector is a single-stranded AAV (ssAAV).

49. The recombinant AAV vector of claim 47, wherein the recombinant AAV vector is a self-complementary AAV (scAAV).

50. The recombinant AAV vector of any one of claims 47-49, wherein the AAV vector comprises a capsid protein of AAV1, AAV2, AAV3, AAV4, AAV5, AAV6, AAV7, AAV8, AAV9, AAV10, AAV11, AAV12, AAVrh8, AAVrh10, AAVrh32.33, AAVrh74, Avian AAV or Bovine AAV.

51. The recombinant AAV vector of any one of claims 47-49, wherein the AAV vector comprises a capsid protein with one or more substitutions or mutations compared to a wildtype AAV capsid protein.

52. A composition comprising the nucleic acid of any one of claims 1-43, the plasmid of claim 44, the cell of claim 45, or the recombinant AAV vector of any one of claims 47-51.

53. A method for treating a subject in need thereof comprising administering to the subject a therapeutically effective amount of the nucleic acid of any one of claims 1-43, the plasmid of claim 44, the cell of claim 45, or the recombinant AAV vector of any one of claims 47-41.

54. The method of claim 53, wherein the subject has Friedreich's Ataxia.

55. The method of claim 53 or 54, wherein the subject is a human subject.

56. The method of any one of claims 53-55, wherein the nucleic acid, the plasmid, the cell, or the recombinant AAV vector is administered by direct injection into the central nervous system.


SEQUENCE LISTING (70 sequences)

SEQ ID NO: 1 - DNA, 141 bp, Dependoparvovirus Adeno-associated virus serotype 2
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 2 - DNA, 113 bp, Artificial Sequence
  3′ ITR
ccactccctctctgcgcgctcgctcgctcactgaggccgggcgaccaaaggtcgcccgacgcccgggctttgcccgggcggcctcagtgagcgagcgagcgcgcagagaggga

SEQ ID NO: 3 - DNA, 141 bp, Artificial Sequence
  3′ ITR
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 4 - DNA, 380 bp, Unknown
  Cytomegalovirus sp.
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatg

SEQ ID NO: 5 - DNA, 286 bp, Unknown
  Cytomegalovirus sp.
cgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatg

SEQ ID NO: 6 - DNA, 276 bp, Gallus gallus
tcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 7 - DNA, 227 bp, Homo sapiens
gcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacgcgtaag

SEQ ID NO: 8 - DNA, 1182 bp, Homo sapiens
gctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 9 - DNA, 212 bp, Artificial Sequence
  EF1a core promoter
gggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacgc

SEQ ID NO: 10 - DNA, 511 bp, Homo sapiens
ggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccag

SEQ ID NO: 11 - DNA, 240 bp, Homo sapiens
cggctggggctgagggtgagggtcccgtttccccaaaggcctagcctggggttccagccacaagccctaccgggcagcgcccggccccgcccctccaggcctggcactcgtcctcaaccaagatggcgcggatggcttcaggcgcatcacgacaccggcgcgtcacgcgacccgccctacgggcacctcccgcgcttttcttagcgccgcagacggtggccgagcgggggaccgggaagc

SEQ ID NO: 12 - DNA, 379 bp, Homo sapiens
attcctgctgggaaaagcaagtggaggtgctccttgaagaaacagggggatcccaccgatctcaggggttctgttctggcctgcggccctggatcgtccagcctgggtcggggtggggagcagacctcgcccttatcggctggggctgagggtgagggtcccgtttccccaaaggcctagcctggggttccagccacaagccctaccgggcagcgcccggccccgcccctccaggcctggcactcgtcctcaaccaagatggcgcggatggcttcaggcgcatcacgacaccggcgcgtcacgcgacccgccctacgggcacctcccgcgcttttcttagcgccgcagacggtggtcgagcgggggaccgggaagctta

SEQ ID NO: 13 - DNA, 229 bp, Artificial Sequence
  Intronic Sequence - Hybrid
ggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcag

SEQ ID NO: 14 - DNA, 476 bp, Homo sapiens
gtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacag

SEQ ID NO: 15 - DNA, 133 bp, Artificial Sequence
  Intronic Sequence - Chimeric
gtaagtatcaaggttacaagacaggtttaaggagaccaatagaaactgggcttgtcgagacagagaagactcttgcgtttctgataggcacctattggtcttactgacatccactttgcctttctctccacag

SEQ ID NO: 16 - DNA, 97 bp, Simian virus 40
gtaagtttagtctttttgtcttttatttcaggtcccggatccggtggtggtgcaaatcaaagaactgctcctcagtggatgttgcctttacttctag

SEQ ID NO: 17 - DNA, 12 bp, Artificial Sequence
  Modified Kozak Sequence
acccggagcagc

SEQ ID NO: 18 - DNA, 9 bp, Artificial Sequence
  Consensus Kozak Sequence
acagccacc

SEQ ID NO: 19 - DNA, 633 bp, Homo sapiens
atgtggactctcgggcgccgcgcagtagccggcctcctggcgtcacccagcccagcccaggcccagaccctcacccgggtcccgcggccggcagagttggccccactctgcggccgccgtggcctgcgcaccgacatcgatgcgacctgcacgccccgccgcgcaagttcgaaccaacgtggcctcaaccagatttggaatgtcaaaaagcagagtgtctatttgatgaatttgaggaaatctggaactttgggccacccaggctctctagatgagaccacctatgaaagactagcagaggaaacgctggactctttagcagagttttttgaagaccttgcagacaagccatacacgtttgaggactatgatgtctcctttgggagtggtgtcttaactgtcaaactgggtggagatctaggaacctatgtgatcaacaagcagacgccaaacaagcaaatctggctatcttctccatccagtggacctaagcgttatgactggactgggaaaaactgggtgtactcccacgacggcgtgtccctccatgagctgctggccgcagagctcactaaagccttaaaaaccaaactggacttgtcttccttggcctattccggaaaagatgcttga

SEQ ID NO: 20 - DNA, 633 bp, Artificial Sequence
  Frataxin cDNA, CpG optimized
atgtggactctggggaggagagcagtagctggcctcctggcatcacccagcccagcccaggcccagaccctcaccagggtccctagaccagcagagttggccccactctgtggcaggagaggcctgaggacagacattgatgccacctgcacccccaggagagcaagttccaaccaaagaggcctcaaccagatttggaatgtcaaaaagcagagtgtctatttgatgaatttgaggaaatctggaactttgggccacccaggctctctagatgagaccacctatgaaagactagcagaggaaacactggactctttagcagagttttttgaagaccttgcagacaagccatacacctttgaggactatgatgtctcctttgggagtggtgtcttaactgtcaaactgggtggagatctaggaacctatgtgatcaacaagcagactccaaacaagcaaatctggctatcttctccatccagtggacctaagaggtatgactggactgggaaaaactgggtgtactcccatgatggagtgtccctccatgagctgctggctgcagagctcactaaagccttaaaaaccaaactggacttgtcttccttggcctattctggaaaagatgcttga

SEQ ID NO: 21 - DNA, 225 bp, Bos taurus
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 22 - DNA, 477 bp, Homo sapiens
gggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtcctt

SEQ ID NO: 23 - DNA, 395 bp, Artificial Sequence
  human beta globin polyadenylation signal (reverse orientation)
taaaatacagcatagcaaaactttaacctccaaatcaagcctctacttgaatccttttctgagggatgaataaggcataggcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagc

SEQ ID NO: 24 - DNA, 56 bp, Oryctolagus cuniculus
aataaaggaaatttattttcattgcaatagtgtgttggaattttttgtgtctctca

SEQ ID NO: 25 - DNA, 133 bp, Artificial Sequence; identical to SEQ ID NO: 15
  chimeric intron stuffer

SEQ ID NO: 26 - DNA, 500 bp, Artificial Sequence
  A1AT stuffer
agagacacagtttttgctctggtgaattacatcttctttaaaggcaaatgggagagaccctttgaagtcaaggacacagaggaagaggacttcctagtggaccaggtgaccaccttgaaggtgcctatgtaaaagcatttaggcatgtttaacatccagcactgtaagaagctgtccagctgggtgctgctgtaaaaatacctgggcaatgccaccaccatcttcttcctgcctgatgaggggaaactacagcacctggaaaatgaactcacccactatattatcaccaagttcctggaaaatgaagacagaaggtctgccagcttacatttacccaaactgtcaattactggaacctatgatctgaagagcttcctgggtcaactgggcatcactaaggtcttcagcaatggggctgacctctcctgggtcacagaggaggcacccctgaagctctccaaggccttgcataaggctgtgctgaccatcaataagaaagg

SEQ ID NO: 27 - DNA, 502 bp, Homo sapiens
taagtttttgtatgaatatgcaagaaggcatcctgattactctgtcttgctgctgctgagacttgccaagacctatgaaaccactctagagaagtgctgtgcctctgcagatcctcatgaatgctatgccaaagtgttcagtgaatttaaacctcttgtggaagagcctcagaatttaatcaaacaaaattgtgagctttttgagcagcttggagagtacaaattccagaatgcactattagttctttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccttggtcctgaaccagttatgtgtgttgcatgagaaaacaccagtaagtgacagagtcaccaaatgctgcacagaatccttggtgaacaggcaaccatgcttttcagctctggaagttgatgaa

SEQ ID NO: 28 - DNA, 2226 bp, Artificial Sequence
  AAV Transfer Cassette
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgcgtcgacaggcctcctaggcggaccgcttgcatgcacgcgttcgcgaagtactcacgtggacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtacccggagcagcatgtggactctcgggcgccgcgcagtagccggcctcctggcgtcacccagcccagcccaggcccagaccctcacccgggtcccgcggccggcagagttggccccactctgcggccgccgtggcctgcgcaccgacatcgatgcgacctgcacgccccgccgcgcaagttcgaaccaacgtggcctcaaccagatttggaatgtcaaaaagcagagtgtctatttgatgaatttgaggaaatctggaactttgggccacccaggctctctagatgagaccacctatgaaagactagcagaggaaacgctggactctttagcagagttttttgaagaccttgcagacaagccatacacgtttgaggactatgatgtctcctttgggagtggtgtcttaactgtcaaactgggtggagatctaggaacctatgtgatcaacaagcagacgccaaacaagcaaatctggctatcttctccatccagtggacctaagcgttatgactggactgggaaaaactgggtgtactcccacgacggcgtgtccctccatgagctgctggccgcagagctcactaaagccttaaaaaccaaactggacttgtcttccttggcctattccggaaaagatgcttgattctaggatccgactgcaggtaggtttaaacaagcttggtaccgtgattaatcttcgaatgactgacctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggagatctgtgtgttggttttttgtgtgcgtacggagctaccaggtctcgagccatgggcgcgccatcgatgactagtccactccctctctgcgcgctcgctcgctcactgaggccgggcgaccaaaggtcgcccgacgcccgggctttgcccgggcggcctcagtgagcgagcgagcgcgcagagaggga

SEQ ID NO: 29 - DNA, 2089 bp, Artificial Sequence
  AAV Transfer Cassette
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgcgtcgacaggcctcctaggcggaccgcttgcatgcacgcgttcgcgaagtactcacgtggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacgcgtaagggcgaattccagcacactggcggccgttactagagccatgcatgtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagcaccggtacccggagcagcatgtggactctcgggcgccgcgcagtagccggcctcctggcgtcacccagcccagcccaggcccagaccctcacccgggtcccgcggccggcagagttggccccactctgcggccgccgtggcctgcgcaccgacatcgatgcgacctgcacgccccgccgcgcaagttcgaaccaacgtggcctcaaccagatttggaatgtcaaaaagcagagtgtctatttgatgaatttgaggaaatctggaactttgggccacccaggctctctagatgagaccacctatgaaagactagcagaggaaacgctggactctttagcagagttttttgaagaccttgcagacaagccatacacgtttgaggactatgatgtctcctttgggagtggtgtcttaactgtcaaactgggtggagatctaggaacctatgtgatcaacaagcagacgccaaacaagcaaatctggctatcttctccatccagtggacctaagcgttatgactggactgggaaaaactgggtgtactcccacgacggcgtgtccctccatgagctgctggccgcagagctcactaaagccttaaaaaccaaactggacttgtcttccttggcctattccggaaaagatgcttgattctaggatccgactgcaggtaggtttaaacaagcttggtaccgtgattaatcttcgaatgactgacctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggagatctgtgtgttggttttttgtgtgcgtacggagctaccaggtctcgagccatgggcgcgccatcgatgactagtccactccctctctgcgcgctcgctcgctcactgaggccgggcgaccaaaggtcgcccgacgcccgggctttgcccgggcggcctcagtgagcgagcgagcgcgcagagaggga

SEQ ID NO: 30 - DNA, 1998 bp, Artificial Sequence
  AAV Transfer Cassette
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgcgtcgacaggcctcctaggcggaccgcttgcatgcacgcgttcgcgaagtactcacgtggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacgcgtaagggcgaattccagcacactggcggccgttactagagccatgcatgtaagtatcaaggttacaagacaggtttaaggagaccaatagaaactgggcttgtcgagacagagaagactcttgcgtttctgataggcacctattggtcttactgacatccactttgcctttctctccacagcaccggtacccggagcagcatgtggactctcgggcgccgcgcagtagccggcctcctggcgtcacccagcccagcccaggcccagaccctcacccgggtcccgcggccggcagagttggccccactctgcggccgccgtggcctgcgcaccgacatcgatgcgacctgcacgccccgccgcgcaagttcgaaccaacgtggcctcaaccagatttggaatgtcaaaaagcagagtgtctatttgatgaatttgaggaaatctggaactttgggccacccaggctctctagatgagaccacctatgaaagactagcagaggaaacgctggactctttagcagagttttttgaagaccttgcagacaagccatacacgtttgaggactatgatgtctcctttgggagtggtgtcttaactgtcaaactgggtggagatctaggaacctatgtgatcaacaagcagacgccaaacaagcaaatctggctatcttctccatccagtggacctaagcgttatgactggactgggaaaaactgggtgtactcccacgacggcgtgtccctccatgagctgctggccgcagagctcactaaagccttaaaaaccaaactggacttgtcttccttggcctattccggaaaagatgcttgattctaggatccgactgcaggtaggtttaaacaagcttggtaccgtgattaatcttcgaatgactgacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttagatctgtgtgttggttttttgtgtgcgtacggagctaccaggtctcgagccatgggcgcgccatcgatgactagtccactccctctctgcgcgctcgctcgctcactgaggccgggcgaccaaaggtcgcccgacgcccgggctttgcccgggcggcctcagtgagcgagcgagcgcgcagagaggga

SEQ ID NO: 31 - DNA, 2133 bp, Artificial Sequence
  AAV Transfer Cassette
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgcgtcgacaggcctcctaggcggaccgcttgcatgcacgcgttcgcgaagtactcacgtgcgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtacccggagcagcatgtggactctcgggcgccgcgcagtagccggcctcctggcgtcacccagcccagcccaggcccagaccctcacccgggtcccgcggccggcagagttggccccactctgcggccgccgtggcctgcgcaccgacatcgatgcgacctgcacgccccgccgcgcaagttcgaaccaacgtggcctcaaccagatttggaatgtcaaaaagcagagtgtctatttgatgaatttgaggaaatctggaactttgggccacccaggctctctagatgagaccacctatgaaagactagcagaggaaacgctggactctttagcagagttttttgaagaccttgcagacaagccatacacgtttgaggactatgatgtctcctttgggagtggtgtcttaactgtcaaactgggtggagatctaggaacctatgtgatcaacaagcagacgccaaacaagcaaatctggctatcttctccatccagtggacctaagcgttatgactggactgggaaaaactgggtgtactcccacgacggcgtgtccctccatgagctgctggccgcagagctcactaaagccttaaaaaccaaactggacttgtcttccttggcctattccggaaaagatgcttgattctaggatccgactgcaggtaggtttaaacaagcttggtaccgtgattaatcttcgaatgactgacctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggagatctgtgtgttggttttttgtgtgcgtacggagctaccaggtctcgagccatgggcgcgccatcgatgactagtccactccctctctgcgcgctcgctcgctcactgaggccgggcgaccaaaggtcgcccgacgcccgggctttgcccgggcggcctcagtgagcgagcgagcgcgcagagaggga

SEQ ID NO: 32 - DNA, 2077 bp, Artificial Sequence
  AAV Transfer Cassette
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgcgtcgacaggcctcctaggcggaccgcttgcatgcacgcgttcgcgaagtactcacgtggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacgcgtaagggcgaattccagcacactggcggccgttactagagccatgcatagtgaaccgtcagatcgccatccagcctccggactctagtgatcaggtactagaggaactgaaaaaccagaaagttcatgtaagtatcaaggttacaagacaggtttaaggagaccaatagaaactgggcttgtcgagacagagaagactcttgcgtttctgataggcacctattggtcttactgacatccactttgcctttctctccacagcaccggtacccggagcagcatgtggactctcgggcgccgcgcagtagccggcctcctggcgtcacccagcccagcccaggcccagaccctcacccgggtcccgcggccggcagagttggccccactctgcggccgccgtggcctgcgcaccgacatcgatgcgacctgcacgccccgccgcgcaagttcgaaccaacgtggcctcaaccagatttggaatgtcaaaaagcagagtgtctatttgatgaatttgaggaaatctggaactttgggccacccaggctctctagatgagaccacctatgaaagactagcagaggaaacgctggactctttagcagagttttttgaagaccttgcagacaagccatacacgtttgaggactatgatgtctcctttgggagtggtgtcttaactgtcaaactgggtggagatctaggaacctatgtgatcaacaagcagacgccaaacaagcaaatctggctatcttctccatccagtggacctaagcgttatgactggactgggaaaaactgggtgtactcccacgacggcgtgtccctccatgagctgctggccgcagagctcactaaagccttaaaaaccaaactggacttgtcttccttggcctattccggaaaagatgcttgattctaggatccgactgcaggtaggtttaaacaagcttggtaccgtgattaatcttcgaatgactgacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttagatctgtgtgttggttttttgtgtgcgtacggagctaccaggtctcgagccatgggcgcgccatcgatgactagtccactccctctctgcgcgctcgctcgctcactgaggccgggcgaccaaaggtcgcccgacgcccgggctttgcccgggcggcctcagtgagcgagcgagcgcgcagagaggga

SEQ ID NO: 33 - DNA, 2246 bp, Artificial Sequence
  AAV Transfer Cassette
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgcgtcgacaggcctcctaggcggaccgcttgcatgcacgcgttcgcgaagtactcacgtggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacgcgtaagggcgaattccagcacactggcggccgttactagagccatgcatagtgaaccgtcagatcgccatccagcctccggactctagtgatcaggtactagaggaactgaaaaaccagaaagttaactggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagtacgtagcggccgcgggcccatatggcccagatctgctagcactagtggcgccgtgaattcaccgcgggcccgatccaccggtacccggagcagcatgtggactctcgggcgccgcgcagtagccggcctcctggcgtcacccagcccagcccaggcccagaccctcacccgggtcccgcggccggcagagttggccccactctgcggccgccgtggcctgcgcaccgacatcgatgcgacctgcacgccccgccgcgcaagttcgaaccaacgtggcctcaaccagatttggaatgtcaaaaagcagagtgtctatttgatgaatttgaggaaatctggaactttgggccacccaggctctctagatgagaccacctatgaaagactagcagaggaaacgctggactctttagcagagttttttgaagaccttgcagacaagccatacacgtttgaggactatgatgtctcctttgggagtggtgtcttaactgtcaaactgggtggagatctaggaacctatgtgatcaacaagcagacgccaaacaagcaaatctggctatcttctccatccagtggacctaagcgttatgactggactgggaaaaactgggtgtactcccacgacggcgtgtccctccatgagctgctggccgcagagctcactaaagccttaaaaaccaaactggacttgtcttccttggcctattccggaaaagatgcttgattctaggatccgactgcaggtaggtttaaacaagcttggtaccgtgattaatcttcgaatgactgacctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggagatctgtgtgttggttttttgtgtgcgtacggagctaccaggtctcgagccatgggcgcgccatcgatgactagtccactccctctctgcgcgctcgctcgctcactgaggccgggcgaccaaaggtcgcccgacgcccgggctttgcccgggcggcctcagtgagcgagcgagcgcgcagagaggga

SEQ ID NO: 34 - DNA, 1914 bp, Artificial Sequence
  AAV Transfer Cassette
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgcgtcgaccggctggggctgagggtgagggtcccgtttccccaaaggcctagcctggggttccagccacaagccctaccgggcagcgcccggccccgcccctccaggcctggcactcgtcctcaaccaagatggcgcggatggcttcaggcgcatcacgacaccggcgcgtcacgcgacccgccctacgggcacctcccgcgcttttcttagcgccgcagacggtggccgagcgggggaccgggaagcatgcatgtaagtatcaaggttacaagacaggtttaaggagaccaatagaaactgggcttgtcgagacagagaagactcttgcgtttctgataggcacctattggtcttactgacatccactttgcctttctctccacagcaccggtacccggagcagcatgtggactctcgggcgccgcgcagtagccggcctcctggcgtcacccagcccagcccaggcccagaccctcacccgggtcccgcggccggcagagttggccccactctgcggccgccgtggcctgcgcaccgacatcgatgcgacctgcacgccccgccgcgcaagttcgaaccaacgtggcctcaaccagatttggaatgtcaaaaagcagagtgtctatttgatgaatttgaggaaatctggaactttgggccacccaggctctctagatgagaccacctatgaaagactagcagaggaaacgctggactctttagcagagttttttgaagaccttgcagacaagccatacacgtttgaggactatgatgtctcctttgggagtggtgtcttaactgtcaaactgggtggagatctaggaacctatgtgatcaacaagcagacgccaaacaagcaaatctggctatcttctccatccagtggacctaagcgttatgactggactgggaaaaactgggtgtactcccacgacggcgtgtccctccatgagctgctggccgcagagctcactaaagccttaaaaaccaaactggacttgtcttccttggcctattccggaaaagatgcttgattctaggatccgactgcaggtaggtttaaacaagcttggtaccgtgattaatcttcgaatgactgacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttagatctgtgtgttggttttttgtgtgcgtacggagctaccaggtctcgagccatgggcgcgccatcgatgactagtccactccctctctgcgcgctcgctcgctcactgaggccgggcgaccaaaggtcgcccgacgcccgggctttgcccgggcggcctcagtgagcgagcgagcgcgcagagaggga

SEQ ID NO: 35 - DNA, 2083 bp, Artificial Sequence
  AAV Transfer Cassette
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgcgtcgaccggctggggctgagggtgagggtcccgtttccccaaaggcctagcctggggttccagccacaagccctaccgggcagcgcccggccccgcccctccaggcctggcactcgtcctcaaccaagatggcgcggatggcttcaggcgcatcacgacaccggcgcgtcacgcgacccgccctacgggcacctcccgcgcttttcttagcgccgcagacggtggccgagcgggggaccgggaagcatgaactggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagtacgtagcggccgcgggcccatatggcccagatctgctagcactagtggcgccgtgaattcaccgcgggcccgatccaccggtacccggagcagcatgtggactctcgggcgccgcgcagtagccggcctcctggcgtcacccagcccagcccaggcccagaccctcacccgggtcccgcggccggcagagttggccccactctgcggccgccgtggcctgcgcaccgacatcgatgcgacctgcacgccccgccgcgcaagttcgaaccaacgtggcctcaaccagatttggaatgtcaaaaagcagagtgtctatttgatgaatttgaggaaatctggaactttgggccacccaggctctctagatgagaccacctatgaaagactagcagaggaaacgctggactctttagcagagttttttgaagaccttgcagacaagccatacacgtttgaggactatgatgtctcctttgggagtggtgtcttaactgtcaaactgggtggagatctaggaacctatgtgatcaacaagcagacgccaaacaagcaaatctggctatcttctccatccagtggacctaagcgttatgactggactgggaaaaactgggtgtactcccacgacggcgtgtccctccatgagctgctggccgcagagctcactaaagccttaaaaaccaaactggacttgtcttccttggcctattccggaaaagatgcttgattctaggatccgactgcaggtaggtttaaacaagcttggtaccgtgattaatcttcgaatgactgacctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggagatctgtgtgttggttttttgtgtgcgtacggagctaccaggtctcgagccatgggcgcgccatcgatgactagtccactccctctctgcgcgctcgctcgctcactgaggccgggcgaccaaaggtcgcccgacgcccgggctttgcccgggcggcctcagtgagcgagcgagcgcgcagagaggga

SEQ ID NO: 36 - DNA, 1879 bp, Artificial Sequence
  AAV Transfer Cassette
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgcgtcgaccggctggggctgagggtgagggtcccgtttccccaaaggcctagcctggggttccagccacaagccctaccgggcagcgcccggccccgcccctccaggcctggcactcgtcctcaaccaagatggcgcggatggcttcaggcgcatcacgacaccggcgcgtcacgcgacccgccctacgggcacctcccgcgcttttcttagcgccgcagacggtggccgagcgggggaccgggaagcatgcatgtaagtttagtctttttgtcttttatttcaggtcccggatccggtggtggtgcaaatcaaagaactgctcctcagtggatgttgcctttacttctagcaccggtacccggagcagcatgtggactctcgggcgccgcgcagtagccggcctcctggcgtcacccagcccagcccaggcccagaccctcacccgggtcccgcggccggcagagttggccccactctgcggccgccgtggcctgcgcaccgacatcgatgcgacctgcacgccccgccgcgcaagttcgaaccaacgtggcctcaaccagatttggaatgtcaaaaagcagagtgtctatttgatgaatttgaggaaatctggaactttgggccacccaggctctctagatgagaccacctatgaaagactagcagaggaaacgctggactctttagcagagttttttgaagaccttgcagacaagccatacacgtttgaggactatgatgtctcctttgggagtggtgtcttaactgtcaaactgggtggagatctaggaacctatgtgatcaacaagcagacgccaaacaagcaaatctggctatcttctccatccagtggacctaagcgttatgactggactgggaaaaactgggtgtactcccacgacggcgtgtccctccatgagctgctggccgcagagctcactaaagccttaaaaaccaaactggacttgtcttccttggcctattccggaaaagatgcttgattctaggatccgactgcaggtaggtttaaacaagcttggtaccgtgattaatcttcgaatgactgacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttagatctgtgtgttggttttttgtgtgcgtacggagctaccaggtctcgagccatggggcgcgccatcgatgactagtccactccctctctgcgcgctcgctcgctcactgaggccgggcgaccaaaggtcgcccgacgcccgggctttgcccgggcggcctcagtgagcgagcgagcgcgcagagaggga

SEQ ID NO: 37 - DNA, 2018 bp, Artificial Sequence
  AAV Transfer Cassette
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgcgtcgacattcctgctgggaaaagcaagtggaggtgctccttgaagaaacagggggatcccaccgatctcaggggttctgttctggcctgcggccctggatcgtccagcctgggtcggggtggggagcagacctcgcccttatcggctggggctgagggtgagggtcccgtttccccaaaggcctagcctggggttccagccacaagccctaccgggcagcgcccggccccgcccctccaggcctggcactcgtcctcaaccaagatggcgcggatggcttcaggcgcatcacgacaccggcgcgtcacgcgacccgccctacgggcacctcccgcgcttttcttagcgccgcagacggtggtcgagcgggggaccgggaagcttaatgcatgtaagtttagtctttttgtcttttatttcaggtcccggatccggtggtggtgcaaatcaaagaactgctcctcagtggatgttgcctttacttctagcaccggtacccggagcagcatgtggactctcgggcgccgcgcagtagccggcctcctggcgtcacccagcccagcccaggcccagaccctcacccgggtcccgcggccggcagagttggccccactctgcggccgccgtggcctgcgcaccgacatcgatgcgacctgcacgccccgccgcgcaagttcgaaccaacgtggcctcaaccagatttggaatgtcaaaaagcagagtgtctatttgatgaatttgaggaaatctggaactttgggccacccaggctctctagatgagaccacctatgaaagactagcagaggaaacgctggactctttagcagagttttttgaagaccttgcagacaagccatacacgtttgaggactatgatgtctcctttgggagtggtgtcttaactgtcaaactgggtggagatctaggaacctatgtgatcaacaagcagacgccaaacaagcaaatctggctatcttctccatccagtggacctaagcgttatgactggactgggaaaaactgggtgtactcccacgacggcgtgtccctccatgagctgctggccgcagagctcactaaagccttaaaaaccaaactggacttgtcttccttggcctattccggaaaagatgcttgattctaggatccgactgcaggtaggtttaaacaagcttggtaccgtgattaatcttcgaatgactgacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttagatctgtgtgttggttttttgtgtgcgtacggagctaccaggtctcgagccatggggcgcgccatcgatgactagtccactccctctctgcgcgctcgctcgctcactgaggccgggcgaccaaaggtcgcccgacgcccgggctttgcccgggcggcctcagtgagcgagcgagcgcgcagagaggga

SEQ ID NO: 38 - DNA, 2134 bp, Artificial Sequence
  AAV Transfer Cassette
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgcgtcgacaggcctcctaggcggaccgcttgcatgcacgcgttcgcgaagtactcacgtgcgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtacccacagccaccatgtggactctcgggcgccgcgcagtagccggcctcctggcgtcacccagcccagcccaggcccagaccctcacccgggtcccgcggccggcagagttggccccactctgcggccgccgtggcctgcgcaccgacatcgatgcgacctgcacgccccgccgcgcaagttcgaaccaacgtggcctcaaccagatttggaatgtcaaaaagcagagtgtctatttgatgaatttgaggaaatctggaactttgggccacccaggctctctagatgagaccacctatgaaagactagcagaggaaacgctggactctttagcagagttttttgaagaccttgcagacaagccatacacgtttgaggactatgatgtctcctttgggagtggtgtcttaactgtcaaactgggtggagatctaggaacctatgtgatcaacaagcagacgccaaacaagcaaatctggctatcttctccatccagtggacctaagcgttatgactggactgggaaaaactgggtgtactcccacgacggcgtgtccctccatgagctgctggccgcagagctcactaaagccttaaaaaccaaactggacttgtcttccttggcctattccggaaaagatgcttgattctaggatccgactgcaggtaggtttaaacaagcttggtaccgtgattaatcttcgaatgactgacctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggagatctgtgtgttggttttttgtgtgcgtacggagctaccaggtctcgagccatgggcgcgccatcgatgactagtccactccctctctgcgcgctcgctcgctcactgaggccgggcgaccaaaggtcgcccgacgcccgggctttgcccgggcggcctcagtgagcgagcgagcgcgcagagaggga

SEQ ID NO: 39 - DNA, 2078 bp, Artificial Sequence
  AAV Transfer Cassette
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgcgtcgacaggcctcctaggcggaccgcttgcatgcacgcgttcgcgaagtactcacgtggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacgcgtaagggcgaattccagcacactggcggccgttactagagccatgcatagtgaaccgtcagatcgccatccagcctccggactctagtgatcaggtactagaggaactgaaaaaccagaaagttcatgtaagtatcaaggttacaagacaggtttaaggagaccaatagaaactgggcttgtcgagacagagaagactcttgcgtttctgataggcacctattggtcttactgacatccactttgcctttctctccacagcaccggtacccacagccaccatgtggactctcgggcgccgcgcagtagccggcctcctggcgtcacccagcccagcccaggcccagaccctcacccgggtcccgcggccggcagagttggccccactctgcggccgccgtggcctgcgcaccgacatcgatgcgacctgcacgccccgccgcgcaagttcgaaccaacgtggcctcaaccagatttggaatgtcaaaaagcagagtgtctatttgatgaatttgaggaaatctggaactttgggccacccaggctctctagatgagaccacctatgaaagactagcagaggaaacgctggactctttagcagagttttttgaagaccttgcagacaagccatacacgtttgaggactatgatgtctcctttgggagtggtgtcttaactgtcaaactgggtggagatctaggaacctatgtgatcaacaagcagacgccaaacaagcaaatctggctatcttctccatccagtggacctaagcgttatgactggactgggaaaaactgggtgtactcccacgacggcgtgtccctccatgagctgctggccgcagagctcactaaagccttaaaaaccaaactggacttgtcttccttggcctattccggaaaagatgcttgattctaggatccgactgcaggtaggtttaaacaagcttggtaccgtgattaatcttcgaatgactgacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttagatctgtgtgttggttttttgtgtgcgtacggagctaccaggtctcgagccatgggcgcgccatcgatgactagtccactccctctctgcgcgctcgctcgctcactgaggccgggcgaccaaaggtcgcccgacgcccgggctttgcccgggcggcctcagtgagcgagcgagcgcgcagagaggga

SEQ ID NO: 40 - DNA, 2247 bp, Artificial Sequence
  AAV Transfer Cassette
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgcgtcgacaggcctcctaggcggaccgcttgcatgcacgcgttcgcgaagtactcacgtggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacgcgtaagggcgaattccagcacactggcggccgttactagagccatgcatagtgaaccgtcagatcgccatccagcctccggactctagtgatcaggtactagaggaactgaaaaaccagaaagttaactggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagtacgtagcggccgcgggcccatatggcccagatctgctagcactagtggcgccgtgaattcaccgcgggcccgatccaccggtacccacagccaccatgtggactctcgggcgccgcgcagtagccggcctcctggcgtcacccagcccagcccaggcccagaccctcacccgggtcccgcggccggcagagttggccccactctgcggccgccgtggcctgcgcaccgacatcgatgcgacctgcacgccccgccgcgcaagttcgaaccaacgtggcctcaaccagatttggaatgtcaaaaagcagagtgtctatttgatgaatttgaggaaatctggaactttgggccacccaggctctctagatgagaccacctatgaaagactagcagaggaaacgctggactctttagcagagttttttgaagaccttgcagacaagccatacacgtttgaggactatgatgtctcctttgggagtggtgtcttaactgtcaaactgggtggagatctaggaacctatgtgatcaacaagcagacgccaaacaagcaaatctggctatcttctccatccagtggacctaagcgttatgactggactgggaaaaactgggtgtactcccacgacggcgtgtccctccatgagctgctggccgcagagctcactaaagccttaaaaaccaaactggacttgtcttccttggcctattccggaaaagatgcttgattctaggatccgactgcaggtaggtttaaacaagcttggtaccgtgattaatcttcgaatgactgacctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggagatctgtgtgttggttttttgtgtgcgtacggagctaccaggtctcgagccatgggcgcgccatcgatgactagtccactccctctctgcgcgctcgctcgctcactgaggccgggcgaccaaaggtcgcccgacgcccgggctttgcccgggcggcctcagtgagcgagcgagcgcgcagagaggga

SEQ ID NO: 41 - DNA, 1915 bp, Artificial Sequence
  AAV Transfer Cassette
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgcgtcgaccggctggggctgagggtgagggtcccgtttccccaaaggcctagcctggggttccagccacaagccctaccgggcagcgcccggccccgcccctccaggcctggcactcgtcctcaaccaagatggcgcggatggcttcaggcgcatcacgacaccggcgcgtcacgcgacccgccctacgggcacctcccgcgcttttcttagcgccgcagacggtggccgagcgggggaccgggaagcatgcatgtaagtatcaaggttacaagacaggtttaaggagaccaatagaaactgggcttgtcgagacagagaagactcttgcgtttctgataggcacctattggtcttactgacatccactttgcctttctctccacagcaccggtacccacagccaccatgtggactctcgggcgccgcgcagtagccggcctcctggcgtcacccagcccagcccaggcccagaccctcacccgggtcccgcggccggcagagttggccccactctgcggccgccgtggcctgcgcaccgacatcgatgcgacctgcacgccccgccgcgcaagttcgaaccaacgtggcctcaaccagatttggaatgtcaaaaagcagagtgtctatttgatgaatttgaggaaatctggaactttgggccacccaggctctctagatgagaccacctatgaaagactagcagaggaaacgctggactctttagcagagttttttgaagaccttgcagacaagccatacacgtttgaggactatgatgtctcctttgggagtggtgtcttaactgtcaaactgggtggagatctaggaacctatgtgatcaacaagcagacgccaaacaagcaaatctggctatcttctccatccagtggacctaagcgttatgactggactgggaaaaactgggtgtactcccacgacggcgtgtccctccatgagctgctggccgcagagctcactaaagccttaaaaaccaaactggacttgtcttccttggcctattccggaaaagatgcttgattctaggatccgactgcaggtaggtttaaacaagcttggtaccgtgattaatcttcgaatgactgacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttagatctgtgtgttggttttttgtgtgcgtacggagctaccaggtctcgagccatgggcgcgccatcgatgactagtccactccctctctgcgcgctcgctcgctcactgaggccgggcgaccaaaggtcgcccgacgcccgggctttgcccgggcggcctcagtgagcgagcgagcgcgcagagaggga

SEQ ID NO: 42 - DNA, 2084 bp, Artificial Sequence
  AAV Transfer Cassette
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgcgtcgaccggctggggctgagggtgagggtcccgtttccccaaaggcctagcctggggttccagccacaagccctaccgggcagcgcccggccccgcccctccaggcctggcactcgtcctcaaccaagatggcgcggatggcttcaggcgcatcacgacaccggcgcgtcacgcgacccgccctacgggcacctcccgcgcttttcttagcgccgcagacggtggccgagcgggggaccgggaagcatgaactggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagtacgtagcggccgcgggcccatatggcccagatctgctagcactagtggcgccgtgaattcaccgcgggcccgatccaccggtacccacagccaccatgtggactctcgggcgccgcgcagtagccggcctcctggcgtcacccagcccagcccaggcccagaccctcacccgggtcccgcggccggcagagttggccccactctgcggccgccgtggcctgcgcaccgacatcgatgcgacctgcacgccccgccgcgcaagttcgaaccaacgtggcctcaaccagatttggaatgtcaaaaagcagagtgtctatttgatgaatttgaggaaatctggaactttgggccacccaggctctctagatgagaccacctatgaaagactagcagaggaaacgctggactctttagcagagttttttgaagaccttgcagacaagccatacacgtttgaggactatgatgtctcctttgggagtggtgtcttaactgtcaaactgggtggagatctaggaacctatgtgatcaacaagcagacgccaaacaagcaaatctggctatcttctccatccagtggacctaagcgttatgactggactgggaaaaactgggtgtactcccacgacggcgtgtccctccatgagctgctggccgcagagctcactaaagccttaaaaaccaaactggacttgtcttccttggcctattccggaaaagatgcttgattctaggatccgactgcaggtaggtttaaacaagcttggtaccgtgattaatcttcgaatgactgacctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggagatctgtgtgttggttttttgtgtgcgtacggagctaccaggtctcgagccatgggcgcgccatcgatgactagtccactccctctctgcgcgctcgctcgctcactgaggccgggcgaccaaaggtcgcccgacgcccgggctttgcccgggcggcctcagtgagcgagcgagcgcgcagagaggga

SEQ ID NO: 43 - DNA, 1880 bp, Artificial Sequence
  AAV Transfer Cassette
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgcgtcgaccggctggggctgagggtgagggtcccgtttccccaaaggcctagcctggggttccagccacaagccctaccgggcagcgcccggccccgcccctccaggcctggcactcgtcctcaaccaagatggcgcggatggcttcaggcgcatcacgacaccggcgcgtcacgcgacccgccctacgggcacctcccgcgcttttcttagcgccgcagacggtggccgagcgggggaccgggaagcatgcatgtaagtttagtctttttgtcttttatttcaggtcccggatccggtggtggtgcaaatcaaagaactgctcctcagtggatgttgcctttacttctagcaccggtacccacagccaccatgtggactctcgggcgccgcgcagtagccggcctcctggcgtcacccagcccagcccaggcccagaccctcacccgggtcccgcggccggcagagttggccccactctgcggccgccgtggcctgcgcaccgacatcgatgcgacctgcacgccccgccgcgcaagttcgaaccaacgtggcctcaaccagatttggaatgtcaaaaagcagagtgtctatttgatgaatttgaggaaatctggaactttgggccacccaggctctctagatgagaccacctatgaaagactagcagaggaaacgctggactctttagcagagttttttgaagaccttgcagacaagccatacacgtttgaggactatgatgtctcctttgggagtggtgtcttaactgtcaaactgggtggagatctaggaacctatgtgatcaacaagcagacgccaaacaagcaaatctggctatcttctccatccagtggacctaagcgttatgactggactgggaaaaactgggtgtactcccacgacggcgtgtccctccatgagctgctggccgcagagctcactaaagccttaaaaaccaaactggacttgtcttccttggcctattccggaaaagatgcttgattctaggatccgactgcaggtaggtttaaacaagcttggtaccgtgattaatcttcgaatgactgacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttagatctgtgtgttggttttttgtgtgcgtacggagctaccaggtctcgagccatggggcgcgccatcgatgactagtccactccctctctgcgcgctcgctcgctcactgaggccgggcgaccaaaggtcgcccgacgcccgggctttgcccgggcggcctcagtgagcgagcgagcgcgcagagaggga

SEQ ID NO: 44 - DNA, 2019 bp, Artificial Sequence
  AAV Transfer Cassette
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgcgtcgacattcctgctgggaaaagcaagtggaggtgctccttgaagaaacagggggatcccaccgatctcaggggttctgttctggcctgcggccctggatcgtccagcctgggtcggggtggggagcagacctcgcccttatcggctggggctgagggtgagggtcccgtttccccaaaggcctagcctggggttccagccacaagccctaccgggcagcgcccggccccgcccctccaggcctggcactcgtcctcaaccaagatggcgcggatggcttcaggcgcatcacgacaccggcgcgtcacgcgacccgccctacgggcacctcccgcgcttttcttagcgccgcagacggtggtcgagcgggggaccgggaagcttaatgcatgtaagtttagtctttttgtcttttatttcaggtcccggatccggtggtggtgcaaatcaaagaactgctcctcagtggatgttgcctttacttctagcaccggtacccacagccaccatgtggactctcgggcgccgcgcagtagccggcctcctggcgtcacccagcccagcccaggcccagaccctcacccgggtcccgcggccggcagagttggccccactctgcggccgccgtggcctgcgcaccgacatcgatgcgacctgcacgccccgccgcgcaagttcgaaccaacgtggcctcaaccagatttggaatgtcaaaaagcagagtgtctatttgatgaatttgaggaaatctggaactttgggccacccaggctctctagatgagaccacctatgaaagactagcagaggaaacgctggactctttagcagagttttttgaagaccttgcagacaagccatacacgtttgaggactatgatgtctcctttgggagtggtgtcttaactgtcaaactgggtggagatctaggaacctatgtgatcaacaagcagacgccaaacaagcaaatctggctatcttctccatccagtggacctaagcgttatgactggactgggaaaaactgggtgtactcccacgacggcgtgtccctccatgagctgctggccgcagagctcactaaagccttaaaaaccaaactggacttgtcttccttggcctattccggaaaagatgcttgattctaggatccgactgcaggtaggtttaaacaagcttggtaccgtgattaatcttcgaatgactgacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttagatctgtgtgttggttttttgtgtgcgtacggagctaccaggtctcgagccatggggcgcgccatcgatgactagtccactccctctctgcgcgctcgctcgctcactgaggccgggcgaccaaaggtcgcccgacgcccgggctttgcccgggcggcctcagtgagcgagcgagcgcgcagagaggga

SEQ ID NO: 45 - DNA, 3063 bp, Artificial Sequence
  AAV Transfer Cassette
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctaacttgcatatgcatgctaaaatacagcatagcaaaactttaacctccaaatcaagcctctacttgaatccttttctgagggatgaataaggcataggcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagcagtactcagtggggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccaggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagcatagcggtacctacccacagccaccatgtggactctcgggcgccgcgcagtagccggcctcctggcgtcacccagcccagcccaggcccagaccctcacccgggtcccgcggccggcagagttggccccactctgcggccgccgtggcctgcgcaccgacatcgatgcgacctgcacgccccgccgcgcaagttcgaaccaacgtggcctcaaccagatttggaatgtcaaaaagcagagtgtctatttgatgaatttgaggaaatctggaactttgggccacccaggctctctagatgagaccacctatgaaagactagcagaggaaacgctggactctttagcagagttttttgaagaccttgcagacaagccatacacgtttgaggactatgatgtctcctttgggagtggtgtcttaactgtcaaactgggtggagatctaggaacctatgtgatcaacaagcagacgccaaacaagcaaatctggctatcttctccatccagtggacctaagcgttatgactggactgggaaaaactgggtgtactcccacgacggcgtgtccctccatgagctgctggccgcagagctcactaaagccttaaaaaccaaactggacttgtcttccttggcctattccggaaaagatgcttgattctaggatgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttagatctgtgtaataaaggaaatttattttcattgcaatagtgtgttggaattttttgtgtctctcagttggttgtaagtatcaaggttacaagacaggtttaaggagaccaatagaaactgggcttgtcgagacagagaagactcttgcgtttctgataggcacctattggtcttactgacatccactttgcctttctctccacagttgtaagctttaccagatgaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 46 - DNA, 3699 bp, Artificial Sequence
  AAV Transfer Cassette
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctaacttgcatatgcatgcagagacacagtttttgctctggtgaattacatcttctttaaaggcaaatgggagagaccctttgaagtcaaggacacagaggaagaggacttcctagtggaccaggtgaccaccttgaaggtgcctatgtaaaagcatttaggcatgtttaacatccagcactgtaagaagctgtccagctgggtgctgctgtaaaaatacctgggcaatgccaccaccatcttcttcctgcctgatgaggggaaactacagcacctggaaaatgaactcacccactatattatcaccaagttcctggaaaatgaagacagaaggtctgccagcttacatttacccaaactgtcaattactggaacctatgatctgaagagcttcctgggtcaactgggcatcactaaggtcttcagcaatggggctgacctctcctgggtcacagaggaggcacccctgaagctctccaaggccttgcataaggctgtgctgaccatcaataagaaaggtaaaatacagcatagcaaaactttaacctccaaatcaagcctctacttgaatccttttctgagggatgaataaggcataggcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagcagtactcagtggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagcatagcggtacctacccacagccaccatgtggactctcgggcgccgcgcagtagccggcctcctggcgtcacccagcccagcccaggcccagaccctcacccgggtcccgcggccggcagagttggccccactctgcggccgccgtggcctgcgcaccgacatcgatgcgacctgcacgccccgccgcgcaagttcgaaccaacgtggcctcaaccagatttggaatgtcaaaaagcagagtgtctatttgatgaatttgaggaaatctggaactttgggccacccaggctctctagatgagaccacctatgaaagactagcagaggaaacgctggactctttagcagagttttttgaagaccttgcagacaagccatacacgtttgaggactatgatgtctcctttgggagtggtgtcttaactgtcaaactgggtggagatctaggaacctatgtgatcaacaagcagacgccaaacaagcaaatctggctatcttctccatccagtggacctaagcgttatgactggactgggaaaaactgggtgtactcccacgacggcgtgtccctccatgagctgctggccgcagagctcactaaagccttaaaaaccaaactggacttgtcttccttggcctattccggaaaagatgcttgattctaggatgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttagatctgtgtaataaaggaaatttattttcattgcaatagtgtgttggaattttttgtgtctctcataagtttttgtatgaatatgcaagaaggcatcctgattactctgtcttgctgctgctgagacttgccaagacctatgaaaccactctagagaagtgctgtgcctctgcagatcctcatgaatgctatgccaaagtgttcagtgaatttaaacctcttgtggaagagcctcagaatttaatcaaacaaaattgtgagctttttgagcagcttggagagtacaaattccagaatgcactattagttctttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccttggtcctgaaccagttatgtgtgttgcatgagaaaacaccagtaagtgacagagtcaccaaatgctgcacagaatccttggtgaacaggcaaccatgcttttcagctctggaagttgatgaagttggttttgtaagctttaccagatgaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 47 - DNA, 3648 bp, Artificial Sequence
  AAV Transfer Cassette
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctaacttgcatatgcatgcagagacacagtttttgctctggtgaattacatcttctttaaaggcaaatgggagagaccctttgaagtcaaggacacagaggaagaggacttcctagtggaccaggtgaccaccttgaaggtgcctatgtaaaagcatttaggcatgtttaacatccagcactgtaagaagctgtccagctgggtgctgctgtaaaaatacctgggcaatgccaccaccatcttcttcctgcctgatgaggggaaactacagcacctggaaaatgaactcacccactatattatcaccaagttcctggaaaatgaagacagaaggtctgccagcttacatttacccaaactgtcaattactggaacctatgatctgaagagcttcctgggtcaactgggcatcactaaggtcttcagcaatggggctgacctctcctgggtcacagaggaggcacccctgaagctctccaaggccttgcataaggctgtgctgaccatcaataagaaaggtaaaatacagcatagcaaaactttaacctccaaatcaagcctctacttgaatccttttctgagggatgaataaggcataggcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagcagtactcagtgggcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacgcgtaaggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagcatagcggtacctacccacagccaccatgtggactctcgggcgccgcgcagtagccggcctcctggcgtcacccagcccagcccaggcccagaccctcacccgggtcccgcggccggcagagttggccccactctgcggccgccgtggcctgcgcaccgacatcgatgcgacctgcacgccccgccgcgcaagttcgaaccaacgtggcctcaaccagatttggaatgtcaaaaagcagagtgtctatttgatgaatttgaggaaatctggaactttgggccacccaggctctctagatgagaccacctatgaaagactagcagaggaaacgctggactctttagcagagttttttgaagaccttgcagacaagccatacacgtttgaggactatgatgtctcctttgggagtggtgtcttaactgtcaaactgggtggagatctaggaacctatgtgatcaacaagcagacgccaaacaagcaaatctggctatcttctccatccagtggacctaagcgttatgactggactgggaaaaactgggtgtactcccacgacggcgtgtccctccatgagctgctggccgcagagctcactaaagccttaaaaaccaaactggacttgtcttccttggcctattccggaaaagatgcttgattctaggatgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttagatctgtgtaataaaggaaatttattttcattgcaatagtgtgttggaattttttgtgtctctcataagtttttgtatgaatatgcaagaaggcatcctgattactctgtcttgctgctgctgagacttgccaagacctatgaaaccactctagagaagtgctgtgcctctgcagatcctcatgaatgctatgccaaagtgttcagtgaatttaaacctcttgtggaagagcctcagaatttaatcaaacaaaattgtgagctttttgagcagcttggagagtacaaattccagaatgcactattagttctttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccttggtcctgaaccagttatgtgtgttgcatgagaaaacaccagtaagtgacagagtcaccaaatgctgcacagaatccttggtgaacaggcaaccatgcttttcagctctggaagttgatgaagttggttttgtaagctttaccagatgaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 48 - DNA, 4127 bp, Artificial Sequence
  AAV Transfer Cassette
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctaacttgcatatgcatgcagagacacagtttttgctctggtgaattacatcttctttaaaggcaaatgggagagaccctttgaagtcaaggacacagaggaagaggacttcctagtggaccaggtgaccaccttgaaggtgcctatgtaaaagcatttaggcatgtttaacatccagcactgtaagaagctgtccagctgggtgctgctgtaaaaatacctgggcaatgccaccaccatcttcttcctgcctgatgaggggaaactacagcacctggaaaatgaactcacccactatattatcaccaagttcctggaaaatgaagacagaaggtctgccagcttacatttacccaaactgtcaattactggaacctatgatctgaagagcttcctgggtcaactgggcatcactaaggtcttcagcaatggggctgacctctcctgggtcacagaggaggcacccctgaagctctccaaggccttgcataaggctgtgctgaccatcaataagaaaggtaaaatacagcatagcaaaactttaacctccaaatcaagcctctacttgaatccttttctgagggatgaataaggcataggcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagcagtactcagtgggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacatagcggtacctacccacagccaccatgtggactctcgggcgccgcgcagtagccggcctcctggcgtcacccagcccagcccaggcccagaccctcacccgggtcccgcggccggcagagttggccccactctgcggccgccgtggcctgcgcaccgacatcgatgcgacctgcacgccccgccgcgcaagttcgaaccaacgtggcctcaaccagatttggaatgtcaaaaagcagagtgtctatttgatgaatttgaggaaatctggaactttgggccacccaggctctctagatgagaccacctatgaaagactagcagaggaaacgctggactctttagcagagttttttgaagaccttgcagacaagccatacacgtttgaggactatgatgtctcctttgggagtggtgtcttaactgtcaaactgggtggagatctaggaacctatgtgatcaacaagcagacgccaaacaagcaaatctggctatcttctccatccagtggacctaagcgttatgactggactgggaaaaactgggtgtactcccacgacggcgtgtccctccatgagctgctggccgcagagctcactaaagccttaaaaaccaaactggacttgtcttccttggcctattccggaaaagatgcttgattctaggatgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttagatctgtgtaataaaggaaatttattttcattgcaatagtgtgttggaattttttgtgtctctcataagtttttgtatgaatatgcaagaaggcatcctgattactctgtcttgctgctgctgagacttgccaagacctatgaaaccactctagagaagtgctgtgcctctgcagatcctcatgaatgctatgccaaagtgttcagtgaatttaaacctcttgtggaagagcctcagaatttaatcaaacaaaattgtgagctttttgagcagcttggagagtacaaattccagaatgcactattagttctttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccttggtcctgaaccagttatgtgtgttgcatgagaaaacaccagtaagtgacagagtcaccaaatgctgcacagaatccttggtgaacaggcaaccatgcttttcagctctggaagttgatgaagttggttttgtaagctttaccagatgaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 49 - DNA, 3932 bp, Artificial Sequence
  AAV Transfer Cassette
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctaacttgcatatgcatgcagagacacagtttttgctctggtgaattacatcttctttaaaggcaaatgggagagaccctttgaagtcaaggacacagaggaagaggacttcctagtggaccaggtgaccaccttgaaggtgcctatgtaaaagcatttaggcatgtttaacatccagcactgtaagaagctgtccagctgggtgctgctgtaaaaatacctgggcaatgccaccaccatcttcttcctgcctgatgaggggaaactacagcacctggaaaatgaactcacccactatattatcaccaagttcctggaaaatgaagacagaaggtctgccagcttacatttacccaaactgtcaattactggaacctatgatctgaagagcttcctgggtcaactgggcatcactaaggtcttcagcaatggggctgacctctcctgggtcacagaggaggcacccctgaagctctccaaggccttgcataaggctgtgctgaccatcaataagaaaggtaaaatacagcatagcaaaactttaacctccaaatcaagcctctacttgaatccttttctgagggatgaataaggcataggcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagcagtactcagtggggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccaggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagcatagcggtacctacccacagccaccatgtggactctcgggcgccgcgcagtagccggcctcctggcgtcacccagcccagcccaggcccagaccctcacccgggtcccgcggccggcagagttggccccactctgcggccgccgtggcctgcgcaccgacatcgatgcgacctgcacgccccgccgcgcaagttcgaaccaacgtggcctcaaccagatttggaatgtcaaaaagcagagtgtctatttgatgaatttgaggaaatctggaactttgggccacccaggctctctagatgagaccacctatgaaagactagcagaggaaacgctggactctttagcagagttttttgaagaccttgcagacaagccatacacgtttgaggactatgatgtctcctttgggagtggtgtcttaactgtcaaactgggtggagatctaggaacctatgtgatcaacaagcagacgccaaacaagcaaatctggctatcttctccatccagtggacctaagcgttatgactggactgggaaaaactgggtgtactcccacgacggcgtgtccctccatgagctgctggccgcagagctcactaaagccttaaaaaccaaactggacttgtcttccttggcctattccggaaaagatgcttgattctaggatgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttagatctgtgtaataaaggaaatttattttcattgcaatagtgtgttggaattttttgtgtctctcataagtttttgtatgaatatgcaagaaggcatcctgattactctgtcttgctgctgctgagacttgccaagacctatgaaaccactctagagaagtgctgtgcctctgcagatcctcatgaatgctatgccaaagtgttcagtgaatttaaacctcttgtggaagagcctcagaatttaatcaaacaaaattgtgagctttttgagcagcttggagagtacaaattccagaatgcactattagttctttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccttggtcctgaaccagttatgtgtgttgcatgagaaaacaccagtaagtgacagagtcaccaaatgctgcacagaatccttggtgaacaggcaaccatgcttttcagctctggaagttgatgaagttggttttgtaagctttaccagatgaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 50 - DNA, 3378 bp, Artificial Sequence
  AAV Transfer Cassette
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctaacttgcatatgcatgctaaaatacagcatagcaaaactttaacctccaaatcaagcctctacttgaatccttttctgagggatgaataaggcataggcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagcagtactcagtgggcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacgcgtaagggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtatgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttactttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattctaaatagcggtacctacccacagccaccatgtggactctcgggcgccgcgcagtagccggcctcctggcgtcacccagcccagcccaggcccagaccctcacccgggtcccgcggccggcagagttggccccactctgcggccgccgtggcctgcgcaccgacatcgatgcgacctgcacgccccgccgcgcaagttcgaaccaacgtggcctcaaccagatttggaatgtcaaaaagcagagtgtctatttgatgaatttgaggaaatctggaactttgggccacccaggctctctagatgagaccacctatgaaagactagcagaggaaacgctggactctttagcagagttttttgaagaccttgcagacaagccatacacgtttgaggactatgatgtctcctttgggagtggtgtcttaactgtcaaactgggtggagatctaggaacctatgtgatcaacaagcagacgccaaacaagcaaatctggctatcttctccatccagtggacctaagcgttatgactggactgggaaaaactgggtgtactcccacgacggcgtgtccctccatgagctgctggccgcagagctcactaaagccttaaaaaccaaactggacttgtcttccttggcctattccggaaaagatgcttgattctaggatgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttagatctgtgtaataaaggaaatttattttcattgcaatagtgtgttggaattttttgtgtctctcagttggttgtaagtatcaaggttacaagacaggtttaaggagaccaatagaaactgggcttgtcgagacagagaagactcttgcgtttctgataggcacctattggtcttactgacatccactttgcctttctctccacagttgtaagctttaccagatgaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 51 - DNA, 2247 bp, Artificial Sequence
  AAV Transfer Cassette
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctaacttgcatatgcatggggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacgcctcagtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagggtacctacccacagccaccatgtggactctggggaggagagcagtagctggcctcctggcatcacccagcccagcccaggcccagaccctcaccagggtccctagaccagcagagttggccccactctgtggcaggagaggcctgaggacagacattgatgccacctgcacccccaggagagcaagttccaaccaaagaggcctcaaccagatttggaatgtcaaaaagcagagtgtctatttgatgaatttgaggaaatctggaactttgggccacccaggctctctagatgagaccacctatgaaagactagcagaggaaacactggactctttagcagagttttttgaagaccttgcagacaagccatacacctttgaggactatgatgtctcctttgggagtggtgtcttaactgtcaaactgggtggagatctaggaacctatgtgatcaacaagcagactccaaacaagcaaatctggctatcttctccatccagtggacctaagaggtatgactggactgggaaaaactgggtgtactcccatgatggagtgtccctccatgagctgctggctgcagagctcactaaagccttaaaaaccaaactggacttgtcttccttggcctattctggaaaagatgcttgataagtttaaacttctaggatgctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagcccctgatgcctatgccttattcatccctcagaaaaggattcaagtagaggcttgatttggaggttaaagttttgctatgctgtattttaagatctgtgtaataaaggaaatttattttcattgcaatagtgtgttggaattttttgtgtctctcagttggttgtaagtatcaaggttacaagacaggtttaaggagaccaatagaaactgggcttgtcgagacagagaagactcttgcgtttctgataggcacctattggtcttactgacatccactttgcctttctctccacagtaagctttaccccactccctctctgcgcgctcgctcgctcactgaggccgggcgaccaaaggtcgcccgacgcccgggctttgcccgggcggcctcagtgagcgagcgagcgcgcagagaggga

SEQ ID NO: 52 - DNA, 2166 bp, Artificial Sequence
  AAV Transfer Cassette
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctaacttgcatatgcatggggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacgcctcagtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagggtacctacccacagccaccatgtggactctggggaggagagcagtagctggcctcctggcatcacccagcccagcccaggcccagaccctcaccagggtccctagaccagcagagttggccccactctgtggcaggagaggcctgaggacagacattgatgccacctgcacccccaggagagcaagttccaaccaaagaggcctcaaccagatttggaatgtcaaaaagcagagtgtctatttgatgaatttgaggaaatctggaactttgggccacccaggctctctagatgagaccacctatgaaagactagcagaggaaacactggactctttagcagagttttttgaagaccttgcagacaagccatacacctttgaggactatgatgtctcctttgggagtggtgtcttaactgtcaaactgggtggagatctaggaacctatgtgatcaacaagcagactccaaacaagcaaatctggctatcttctccatccagtggacctaagaggtatgactggactgggaaaaactgggtgtactcccatgatggagtgtccctccatgagctgctggctgcagagctcactaaagccttaaaaaccaaactggacttgtcttccttggcctattctggaaaagatgcttgataacgactgcaggtaggtttaaacaagcttggtaccgtgattaatcttcgaatgactgacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttagataagctttaccccactccctctctgcgcgctcgctcgctcactgaggccgggcgaccaaaggtcgcccgacgcccgggctttgcccgggcggcctcagtgagcgagcgagcgcgcagagaggga

SEQ ID NO: 53 - DNA, 3062 bp, Artificial Sequence
  AAV Transfer Cassette
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctaacttgcatatgcatgctaaaatacagcatagcaaaactttaacctccaaatcaagcctctacttgaatccttttctgagggatgaataaggcataggcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagcagtactcagtggggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccaggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagcatagcggtacctacccggagcagcatgtggactctcgggcgccgcgcagtagccggcctcctggcgtcacccagcccagcccaggcccagaccctcacccgggtcccgcggccggcagagttggccccactctgcggccgccgtggcctgcgcaccgacatcgatgcgacctgcacgccccgccgcgcaagttcgaaccaacgtggcctcaaccagatttggaatgtcaaaaagcagagtgtctatttgatgaatttgaggaaatctggaactttgggccacccaggctctctagatgagaccacctatgaaagactagcagaggaaacgctggactctttagcagagttttttgaagaccttgcagacaagccatacacgtttgaggactatgatgtctcctttgggagtggtgtcttaactgtcaaactgggtggagatctaggaacctatgtgatcaacaagcagacgccaaacaagcaaatctggctatcttctccatccagtggacctaagcgttatgactggactgggaaaaactgggtgtactcccacgacggcgtgtccctccatgagctgctggccgcagagctcactaaagccttaaaaaccaaactggacttgtcttccttggcctattccggaaaagatgcttgattctaggatgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttagatctgtgtaataaaggaaatttattttcattgcaatagtgtgttggaattttttgtgtctctcagttggttgtaagtatcaaggttacaagacaggtttaaggagaccaatagaaactgggcttgtcgagacagagaagactcttgcgtttctgataggcacctattggtcttactgacatccactttgcctttctctccacagttgtaagctttaccagatgaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 54 - DNA, 3698 bp, Artificial Sequence
  AAV Transfer Cassette
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctaacttgcatatgcatgcagagacacagtttttgctctggtgaattacatcttctttaaaggcaaatgggagagaccctttgaagtcaaggacacagaggaagaggacttcctagtggaccaggtgaccaccttgaaggtgcctatgtaaaagcatttaggcatgtttaacatccagcactgtaagaagctgtccagctgggtgctgctgtaaaaatacctgggcaatgccaccaccatcttcttcctgcctgatgaggggaaactacagcacctggaaaatgaactcacccactatattatcaccaagttcctggaaaatgaagacagaaggtctgccagcttacatttacccaaactgtcaattactggaacctatgatctgaagagcttcctgggtcaactgggcatcactaaggtcttcagcaatggggctgacctctcctgggtcacagaggaggcacccctgaagctctccaaggccttgcataaggctgtgctgaccatcaataagaaaggtaaaatacagcatagcaaaactttaacctccaaatcaagcctctacttgaatccttttctgagggatgaataaggcataggcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagcagtactcagtggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagcatagcggtacctacccggagcagcatgtggactctcgggcgccgcgcagtagccggcctcctggcgtcacccagcccagcccaggcccagaccctcacccgggtcccgcggccggcagagttggccccactctgcggccgccgtggcctgcgcaccgacatcgatgcgacctgcacgccccgccgcgcaagttcgaaccaacgtggcctcaaccagatttggaatgtcaaaaagcagagtgtctatttgatgaatttgaggaaatctggaactttgggccacccaggctctctagatgagaccacctatgaaagactagcagaggaaacgctggactctttagcagagttttttgaagaccttgcagacaagccatacacgtttgaggactatgatgtctcctttgggagtggtgtcttaactgtcaaactgggtggagatctaggaacctatgtgatcaacaagcagacgccaaacaagcaaatctggctatcttctccatccagtggacctaagcgttatgactggactgggaaaaactgggtgtactcccacgacggcgtgtccctccatgagctgctggccgcagagctcactaaagccttaaaaaccaaactggacttgtcttccttggcctattccggaaaagatgcttgattctaggatgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttagatctgtgtaataaaggaaatttattttcattgcaatagtgtgttggaattttttgtgtctctcataagtttttgtatgaatatgcaagaaggcatcctgattactctgtcttgctgctgctgagacttgccaagacctatgaaaccactctagagaagtgctgtgcctctgcagatcctcatgaatgctatgccaaagtgttcagtgaatttaaacctcttgtggaagagcctcagaatttaatcaaacaaaattgtgagctttttgagcagcttggagagtacaaattccagaatgcactattagttctttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccttggtcctgaaccagttatgtgtgttgcatgagaaaacaccagtaagtgacagagtcaccaaatgctgcacagaatccttggtgaacaggcaaccatgcttttcagctctggaagttgatgaagttggttttgtaagctttaccagatgaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 55 - DNA, 3647 bp, Artificial Sequence
  AAV Transfer Cassette
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctaacttgcatatgcatgcagagacacagtttttgctctggtgaattacatcttctttaaaggcaaatgggagagaccctttgaagtcaaggacacagaggaagaggacttcctagtggaccaggtgaccaccttgaaggtgcctatgtaaaagcatttaggcatgtttaacatccagcactgtaagaagctgtccagctgggtgctgctgtaaaaatacctgggcaatgccaccaccatcttcttcctgcctgatgaggggaaactacagcacctggaaaatgaactcacccactatattatcaccaagttcctggaaaatgaagacagaaggtctgccagcttacatttacccaaactgtcaattactggaacctatgatctgaagagcttcctgggtcaactgggcatcactaaggtcttcagcaatggggctgacctctcctgggtcacagaggaggcacccctgaagctctccaaggccttgcataaggctgtgctgaccatcaataagaaaggtaaaatacagcatagcaaaactttaacctccaaatcaagcctctacttgaatccttttctgagggatgaataaggcataggcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagcagtactcagtgggcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacgcgtaaggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagcatagcggtacctacccggagcagcatgtggactctcgggcgccgcgcagtagccggcctcctggcgtcacccagcccagcccaggcccagaccctcacccgggtcccgcggccggcagagttggccccactctgcggccgccgtggcctgcgcaccgacatcgatgcgacctgcacgccccgccgcgcaagttcgaaccaacgtggcctcaaccagatttggaatgtcaaaaagcagagtgtctatttgatgaatttgaggaaatctggaactttgggccacccaggctctctagatgagaccacctatgaaagactagcagaggaaacgctggactctttagcagagttttttgaagaccttgcagacaagccatacacgtttgaggactatgatgtctcctttgggagtggtgtcttaactgtcaaactgggtggagatctaggaacctatgtgatcaacaagcagacgccaaacaagcaaatctggctatcttctccatccagtggacctaagcgttatgactggactgggaaaaactgggtgtactcccacgacggcgtgtccctccatgagctgctggccgcagagctcactaaagccttaaaaaccaaactggacttgtcttccttggcctattccggaaaagatgcttgattctaggatgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttagatctgtgtaataaaggaaatttattttcattgcaatagtgtgttggaattttttgtgtctctcataagtttttgtatgaatatgcaagaaggcatcctgattactctgtcttgctgctgctgagacttgccaagacctatgaaaccactctagagaagtgctgtgcctctgcagatcctcatgaatgctatgccaaagtgttcagtgaatttaaacctcttgtggaagagcctcagaatttaatcaaacaaaattgtgagctttttgagcagcttggagagtacaaattccagaatgcactattagttctttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccttggtcctgaaccagttatgtgtgttgcatgagaaaacaccagtaagtgacagagtcaccaaatgctgcacagaatccttggtgaacaggcaaccatgcttttcagctctggaagttgatgaagttggttttgtaagctttaccagatgaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 56 - DNA, 4126 bp, Artificial Sequence
  AAV Transfer Cassette
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctaacttgcatatgcatgcagagacacagtttttgctctggtgaattacatcttctttaaaggcaaatgggagagaccctttgaagtcaaggacacagaggaagaggacttcctagtggaccaggtgaccaccttgaaggtgcctatgtaaaagcatttaggcatgtttaacatccagcactgtaagaagctgtccagctgggtgctgctgtaaaaatacctgggcaatgccaccaccatcttcttcctgcctgatgaggggaaactacagcacctggaaaatgaactcacccactatattatcaccaagttcctggaaaatgaagacagaaggtctgccagcttacatttacccaaactgtcaattactggaacctatgatctgaagagcttcctgggtcaactgggcatcactaaggtcttcagcaatggggctgacctctcctgggtcacagaggaggcacccctgaagctctccaaggccttgcataaggctgtgctgaccatcaataagaaaggtaaaatacagcatagcaaaactttaacctccaaatcaagcctctacttgaatccttttctgagggatgaataaggcataggcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagcagtactcagtgggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacatagcggtacctacccggagcagcatgtggactctcgggcgccgcgcagtagccggcctcctggcgtcacccagcccagcccaggcccagaccctcacccgggtcccgcggccggcagagttggccccactctgcggccgccgtggcctgcgcaccgacatcgatgcgacctgcacgccccgccgcgcaagttcgaaccaacgtggcctcaaccagatttggaatgtcaaaaagcagagtgtctatttgatgaatttgaggaaatctggaactttgggccacccaggctctctagatgagaccacctatgaaagactagcagaggaaacgctggactctttagcagagttttttgaagaccttgcagacaagccatacacgtttgaggactatgatgtctcctttgggagtggtgtcttaactgtcaaactgggtggagatctaggaacctatgtgatcaacaagcagacgccaaacaagcaaatctggctatcttctccatccagtggacctaagcgttatgactggactgggaaaaactgggtgtactcccacgacggcgtgtccctccatgagctgctggccgcagagctcactaaagccttaaaaaccaaactggacttgtcttccttggcctattccggaaaagatgcttgattctaggatgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttagatctgtgtaataaaggaaatttattttcattgcaatagtgtgttggaattttttgtgtctctcataagtttttgtatgaatatgcaagaaggcatcctgattactctgtcttgctgctgctgagacttgccaagacctatgaaaccactctagagaagtgctgtgcctctgcagatcctcatgaatgctatgccaaagtgttcagtgaatttaaacctcttgtggaagagcctcagaatttaatcaaacaaaattgtgagctttttgagcagcttggagagtacaaattccagaatgcactattagttctttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccttggtcctgaaccagttatgtgtgttgcatgagaaaacaccagtaagtgacagagtcaccaaatgctgcacagaatccttggtgaacaggcaaccatgcttttcagctctggaagttgatgaagttggttttgtaagctttaccagatgaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 57 - DNA, 3931 bp, Artificial Sequence
  AAV Transfer Cassette
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctaacttgcatatgcatgcagagacacagtttttgctctggtgaattacatcttctttaaaggcaaatgggagagaccctttgaagtcaaggacacagaggaagaggacttcctagtggaccaggtgaccaccttgaaggtgcctatgtaaaagcatttaggcatgtttaacatccagcactgtaagaagctgtccagctgggtgctgctgtaaaaatacctgggcaatgccaccaccatcttcttcctgcctgatgaggggaaactacagcacctggaaaatgaactcacccactatattatcaccaagttcctggaaaatgaagacagaaggtctgccagcttacatttacccaaactgtcaattactggaacctatgatctgaagagcttcctgggtcaactgggcatcactaaggtcttcagcaatggggctgacctctcctgggtcacagaggaggcacccctgaagctctccaaggccttgcataaggctgtgctgaccatcaataagaaaggtaaaatacagcatagcaaaactttaacctccaaatcaagcctctacttgaatccttttctgagggatgaataaggcataggcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagcagtactcagtggggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccaggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagcatagcggtacctacccggagcagcatgtggactctcgggcgccgcgcagtagccggcctcctggcgtcacccagcccagcccaggcccagaccctcacccgggtcccgcggccggcagagttggccccactctgcggccgccgtggcctgcgcaccgacatcgatgcgacctgcacgccccgccgcgcaagttcgaaccaacgtggcctcaaccagatttggaatgtcaaaaagcagagtgtctatttgatgaatttgaggaaatctggaactttgggccacccaggctctctagatgagaccacctatgaaagactagcagaggaaacgctggactctttagcagagttttttgaagaccttgcagacaagccatacacgtttgaggactatgatgtctcctttgggagtggtgtcttaactgtcaaactgggtggagatctaggaacctatgtgatcaacaagcagacgccaaacaagcaaatctggctatcttctccatccagtggacctaagcgttatgactggactgggaaaaactgggtgtactcccacgacggcgtgtccctccatgagctgctggccgcagagctcactaaagccttaaaaaccaaactggacttgtcttccttggcctattccggaaaagatgcttgattctaggatgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttagatctgtgtaataaaggaaatttattttcattgcaatagtgtgttggaattttttgtgtctctcataagtttttgtatgaatatgcaagaaggcatcctgattactctgtcttgctgctgctgagacttgccaagacctatgaaaccactctagagaagtgctgtgcctctgcagatcctcatgaatgctatgccaaagtgttcagtgaatttaaacctcttgtggaagagcctcagaatttaatcaaacaaaattgtgagctttttgagcagcttggagagtacaaattccagaatgcactattagttctttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccttggtcctgaaccagttatgtgtgttgcatgagaaaacaccagtaagtgacagagtcaccaaatgctgcacagaatccttggtgaacaggcaaccatgcttttcagctctggaagttgatgaagttggttttgtaagctttaccagatgaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 58 - DNA, 3377 bp, Artificial Sequence
  AAV Transfer Cassette
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctaacttgcatatgcatgctaaaatacagcatagcaaaactttaacctccaaatcaagcctctacttgaatccttttctgagggatgaataaggcataggcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagcagtactcagtgggcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacgcgtaagggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtatgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttactttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattctaaatagcggtacctacccggagcagcatgtggactctcgggcgccgcgcagtagccggcctcctggcgtcacccagcccagcccaggcccagaccctcacccgggtcccgcggccggcagagttggccccactctgcggccgccgtggcctgcgcaccgacatcgatgcgacctgcacgccccgccgcgcaagttcgaaccaacgtggcctcaaccagatttggaatgtcaaaaagcagagtgtctatttgatgaatttgaggaaatctggaactttgggccacccaggctctctagatgagaccacctatgaaagactagcagaggaaacgctggactctttagcagagttttttgaagaccttgcagacaagccatacacgtttgaggactatgatgtctcctttgggagtggtgtcttaactgtcaaactgggtggagatctaggaacctatgtgatcaacaagcagacgccaaacaagcaaatctggctatcttctccatccagtggacctaagcgttatgactggactgggaaaaactgggtgtactcccacgacggcgtgtccctccatgagctgctggccgcagagctcactaaagccttaaaaaccaaactggacttgtcttccttggcctattccggaaaagatgcttgattctaggatgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttagatctgtgtaataaaggaaatttattttcattgcaatagtgtgttggaattttttgtgtctctcagttggttgtaagtatcaaggttacaagacaggtttaaggagaccaatagaaactgggcttgtcgagacagagaagactcttgcgtttctgataggcacctattggtcttactgacatccactttgcctttctctccacagttgtaagctttaccagatgaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 59 - DNA, 2246 bp, Artificial Sequence
  AAV Transfer Cassette
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctaacttgcatatgcatggggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacgcctcagtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagggtacctacccggagcagcatgtggactctggggaggagagcagtagctggcctcctggcatcacccagcccagcccaggcccagaccctcaccagggtccctagaccagcagagttggccccactctgtggcaggagaggcctgaggacagacattgatgccacctgcacccccaggagagcaagttccaaccaaagaggcctcaaccagatttggaatgtcaaaaagcagagtgtctatttgatgaatttgaggaaatctggaactttgggccacccaggctctctagatgagaccacctatgaaagactagcagaggaaacactggactctttagcagagttttttgaagaccttgcagacaagccatacacctttgaggactatgatgtctcctttgggagtggtgtcttaactgtcaaactgggtggagatctaggaacctatgtgatcaacaagcagactccaaacaagcaaatctggctatcttctccatccagtggacctaagaggtatgactggactgggaaaaactgggtgtactcccatgatggagtgtccctccatgagctgctggctgcagagctcactaaagccttaaaaaccaaactggacttgtcttccttggcctattctggaaaagatgcttgataagtttaaacttctaggatgctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagcccctgatgcctatgccttattcatccctcagaaaaggattcaagtagaggcttgatttggaggttaaagttttgctatgctgtattttaagatctgtgtaataaaggaaatttattttcattgcaatagtgtgttggaattttttgtgtctctcagttggttgtaagtatcaaggttacaagacaggtttaaggagaccaatagaaactgggcttgtcgagacagagaagactcttgcgtttctgataggcacctattggtcttactgacatccactttgcctttctctccacagtaagctttaccccactccctctctgcgcgctcgctcgctcactgaggccgggcgaccaaaggtcgcccgacgcccgggctttgcccgggcggcctcagtgagcgagcgagcgcgcagagaggga

SEQ ID NO: 60 - DNA, 2165 bp, Artificial Sequence
  AAV Transfer Cassette
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctaacttgcatatgcatggggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacgcctcagtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagggtacctacccggagcagcatgtggactctggggaggagagcagtagctggcctcctggcatcacccagcccagcccaggcccagaccctcaccagggtccctagaccagcagagttggccccactctgtggcaggagaggcctgaggacagacattgatgccacctgcacccccaggagagcaagttccaaccaaagaggcctcaaccagatttggaatgtcaaaaagcagagtgtctatttgatgaatttgaggaaatctggaactttgggccacccaggctctctagatgagaccacctatgaaagactagcagaggaaacactggactctttagcagagttttttgaagaccttgcagacaagccatacacctttgaggactatgatgtctcctttgggagtggtgtcttaactgtcaaactgggtggagatctaggaacctatgtgatcaacaagcagactccaaacaagcaaatctggctatcttctccatccagtggacctaagaggtatgactggactgggaaaaactgggtgtactcccatgatggagtgtccctccatgagctgctggctgcagagctcactaaagccttaaaaaccaaactggacttgtcttccttggcctattctggaaaagatgcttgataacgactgcaggtaggtttaaacaagcttggtaccgtgattaatcttcgaatgactgacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttagataagctttaccccactccctctctgcgcgctcgctcgctcactgaggccgggcgaccaaaggtcgcccgacgcccgggctttgcccgggcggcctcagtgagcgagcgagcgcgcagagaggga

SEQ ID NO: 61 - DNA, 2071 bp, Artificial Sequence
  AAV Transfer Cassette
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtgttgacagacctcctaggaggactgcttgtatgcatgcttttgctaagtactcaagaggttctggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacgcgtaagggctaattccagcacactggctgctgttactagagccatgcatagtgaaccttcagatagacatccagcctctggactctagtgatcaggtactagaggaactgaaaaaccagaaagttcatgtaagtatcaaggttacaagacaggtttaaggagaccaatagaaactgggcttgtcgagacagagaagactcttgcgtttctgataggcacctattggtcttactgacatccactttgcctttctctccacagtactggtacccggagcagcatgtggactctcgggcgccgcgcagtagccggcctcctggcgtcacccagcccagcccaggcccagaccctcacccgggtcccgcggccggcagagttggccccactctgcggccgccgtggcctgcgcaccgacatcgatgcgacctgcacgccccgccgcgcaagttcgaaccaacgtggcctcaaccagatttggaatgtcaaaaagcagagtgtctatttgatgaatttgaggaaatctggaactttgggccacccaggctctctagatgagaccacctatgaaagactagcagaggaaacgctggactctttagcagagttttttgaagaccttgcagacaagccatacacgtttgaggactatgatgtctcctttgggagtggtgtcttaactgtcaaactgggtggagatctaggaacctatgtgatcaacaagcagacgccaaacaagcaaatctggctatcttctccatccagtggacctaagcgttatgactggactgggaaaaactgggtgtactcccacgacggcgtgtccctccatgagctgctggccgcagagctcactaaagccttaaaaaccaaactggacttgtcttccttggcctattccggaaaagatgcttgagatccgactgcaggtaggtttaaacaagcttggtaccgtgattaatcttcgaatgactgacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttagatctgtgtgttggttttttgtgtgagaacagaactaccaggtttagaaccatgggcaagacatagatgactagtccactccctctctgcgcgctcgctcgctcactgaggccgggcgaccaaaggtcgcccgacgcccgggctttgcccgggcggcctcagtgagcgagcgagcgcgcagagaggga

SEQ ID NO: 62 - DNA, 2071 bp, Artificial Sequence
  AAV Transfer Cassette
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtgttgacagacctcctaggaggactgcttgtatgcatgcttttgctaagtactcaagaggttctggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacgcgtaagggctaattccagcacactggctgctgttactagagccatgcatagtgaaccttcagatagacatccagcctctggactctagtgatcaggtactagaggaactgaaaaaccagaaagttcatgtaagtatcaaggttacaagacaggtttaaggagaccaatagaaactgggcttgtcgagacagagaagactcttgcgtttctgataggcacctattggtcttactgacatccactttgcctttctctccacagtactggtacccggagcagcatgtggactctggggaggagagcagtagctggcctcctggcatcacccagcccagcccaggcccagaccctcaccagggtccctagaccagcagagttggccccactctgtggcaggagaggcctgaggacagacattgatgccacctgcacccccaggagagcaagttccaaccaaagaggcctcaaccagatttggaatgtcaaaaagcagagtgtctatttgatgaatttgaggaaatctggaactttgggccacccaggctctctagatgagaccacctatgaaagactagcagaggaaacactggactctttagcagagttttttgaagaccttgcagacaagccatacacctttgaggactatgatgtctcctttgggagtggtgtcttaactgtcaaactgggtggagatctaggaacctatgtgatcaacaagcagactccaaacaagcaaatctggctatcttctccatccagtggacctaagaggtatgactggactgggaaaaactgggtgtactcccatgatggagtgtccctccatgagctgctggctgcagagctcactaaagccttaaaaaccaaactggacttgtcttccttggcctattctggaaaagatgcttgagatccgactgcaggtaggtttaaacaagcttggtaccgtgattaatcttcgaatgactgacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttagatctgtgtgttggttttttgtgtgagaacagaactaccaggtttagaaccatgggcaagacatagatgactagtccactccctctctgcgcgctcgctcgctcactgaggccgggcgaccaaaggtcgcccgacgcccgggctttgcccgggcggcctcagtgagcgagcgagcgcgcagagaggga

SEQ ID NO: 63 - DNA, 2072 bp, Artificial Sequence
  AAV Transfer Cassette
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtgttgacagacctcctaggaggactgcttgtatgcatgcttttgctaagtactcaagaggttctggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacgcgtaagggctaattccagcacactggctgctgttactagagccatgcatagtgaaccttcagatagacatccagcctctggactctagtgatcaggtactagaggaactgaaaaaccagaaagttcatgtaagtatcaaggttacaagacaggtttaaggagaccaatagaaactgggcttgtcgagacagagaagactcttgcgtttctgataggcacctattggtcttactgacatccactttgcctttctctccacagtactggtacccacagccaccatgtggactctcgggcgccgcgcagtagccggcctcctggcgtcacccagcccagcccaggcccagaccctcacccgggtcccgcggccggcagagttggccccactctgcggccgccgtggcctgcgcaccgacatcgatgcgacctgcacgccccgccgcgcaagttcgaaccaacgtggcctcaaccagatttggaatgtcaaaaagcagagtgtctatttgatgaatttgaggaaatctggaactttgggccacccaggctctctagatgagaccacctatgaaagactagcagaggaaacgctggactctttagcagagttttttgaagaccttgcagacaagccatacacgtttgaggactatgatgtctcctttgggagtggtgtcttaactgtcaaactgggtggagatctaggaacctatgtgatcaacaagcagacgccaaacaagcaaatctggctatcttctccatccagtggacctaagcgttatgactggactgggaaaaactgggtgtactcccacgacggcgtgtccctccatgagctgctggccgcagagctcactaaagccttaaaaaccaaactggacttgtcttccttggcctattccggaaaagatgcttgagatccgactgcaggtaggtttaaacaagcttggtaccgtgattaatcttcgaatgactgacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttagatctgtgtgttggttttttgtgtgagaacagaactaccaggtttagaaccatgggcaagacatagatgactagtccactccctctctgcgcgctcgctcgctcactgaggccgggcgaccaaaggtcgcccgacgcccgggctttgcccgggcggcctcagtgagcgagcgagcgcgcagagaggga

SEQ ID NO: 64 - DNA, 2072 bp, Artificial Sequence
  AAV Transfer Cassette
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtgttgacagacctcctaggaggactgcttgtatgcatgcttttgctaagtactcaagaggttctggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacgcgtaagggctaattccagcacactggctgctgttactagagccatgcatagtgaaccttcagatagacatccagcctctggactctagtgatcaggtactagaggaactgaaaaaccagaaagttcatgtaagtatcaaggttacaagacaggtttaaggagaccaatagaaactgggcttgtcgagacagagaagactcttgcgtttctgataggcacctattggtcttactgacatccactttgcctttctctccacagtactggtacccacagccaccatgtggactctggggaggagagcagtagctggcctcctggcatcacccagcccagcccaggcccagaccctcaccagggtccctagaccagcagagttggccccactctgtggcaggagaggcctgaggacagacattgatgccacctgcacccccaggagagcaagttccaaccaaagaggcctcaaccagatttggaatgtcaaaaagcagagtgtctatttgatgaatttgaggaaatctggaactttgggccacccaggctctctagatgagaccacctatgaaagactagcagaggaaacactggactctttagcagagttttttgaagaccttgcagacaagccatacacctttgaggactatgatgtctcctttgggagtggtgtcttaactgtcaaactgggtggagatctaggaacctatgtgatcaacaagcagactccaaacaagcaaatctggctatcttctccatccagtggacctaagaggtatgactggactgggaaaaactgggtgtactcccatgatggagtgtccctccatgagctgctggctgcagagctcactaaagccttaaaaaccaaactggacttgtcttccttggcctattctggaaaagatgcttgagatccgactgcaggtaggtttaaacaagcttggtaccgtgattaatcttcgaatgactgacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttagatctgtgtgttggttttttgtgtgagaacagaactaccaggtttagaaccatgggcaagacatagatgactagtccactccctctctgcgcgctcgctcgctcactgaggccgggcgaccaaaggtcgcccgacgcccgggctttgcccgggcggcctcagtgagcgagcgagcgcgcagagaggga

SEQ ID NO: 65 - protein, 210 aa, Homo sapiens
MWTLGRRAVAGLLASPSPAQAQTLTRVPRPAELAPLCGRRGLRTDIDATCTPRRASSNQRGLNQIWNVKKQSVYLMNLRKSGTLGHPGSLDETTYERLAEETLDSLAEFFEDLADKPYTFEDYDVSFGSGVLTVKLGGDLGTYVINKQTPNKQIWLSSPSSGPKRYDWTGKNWVYSHDGVSLHELLAAELTKALKTKLDLSSLAYSGKDA

SEQ ID NO: 66 - DNA, 7 bp, Artificial Sequence
  Kozak sequence
accatgg

SEQ ID NO: 67 - DNA, 13 bp, Artificial Sequence
  Kozak sequence
gccgccaccatgg

SEQ ID NO: 68 - DNA, 13 bp, Artificial Sequence
  Kozak sequence
gccgccgccatgg

SEQ ID NO: 69 - DNA, 9 bp, Artificial Sequence
  Kozak sequence
ccaccatgg

SEQ ID NO: 70 - DNA, 8 bp, Artificial Sequence
  Kozak sequence
ccaccatg